(19) United States
(12) Patent Application Publication
(10) Pub. No.: US 2008/0274458 A1
(43) Pub. Date: Nov. 6, 2008

(54) NUCLEIC ACID QUANTITATION METHODS

(76) Inventors: Gary J. Latham, Austin, TX (US); Heidi J. Peltier, Austin, TX (US); Jon Kemppainen, Austin, TX (US); Timothy S. Davison, Austin, TX (US); Emmanuel Labourier, Austin, TX (US); David Brown, Austin, TX (US); Matthew Winkler, Austin, TX (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/743,036

(22) Filed: May 1, 2007

(51) Int. Cl. C12Q 1/68; G01N 33/48

(52) U.S. Cl. 435/6; 436/94

(57) ABSTRACT

The invention relates to a method of determining the amount of a target nucleic acid sequence in a sample, the method comprising: obtaining multiple distinguishable amplicons of the target nucleic acid sequence, each comprising a distinguishing tag and a target portion; amplifying the amplicons in a single reaction volume; and detecting nucleic acids amplified from the amplicons. Detection of the distinguishable amplicons can be varied in each of the steps of the method, which expands the dynamic range of the nucleic acid quantification methods and improves the reliability and accuracy of the methods.

### BACKGROUND

The basis of most nucleic acid detection and quantification is hybridization of a detectable probe that reports the amount of the nucleic acid target. A limitation of many nucleic acid detection technologies is the dynamic range of detection in the assays, with many technologies capable of detecting nucleic acids over only two to three orders of magnitude. The amount of a nucleic acid in different samples will often vary over a broader range than the detection limits of the nucleic acid detection assay, and two or more nucleic acids may be found in widely varying abundance within a single sample. Since quantification of a nucleic acid is possible only in a portion of the detectable range of an assay, the linear dynamic range and the overall dynamic range of detection of a nucleic acid detection assay may limit its application in research and diagnostic uses.

Additionally, dynamic range limitations are particularly problematic in nucleic acid detection assays involving nucleic acid amplification. For example, PCR (polymerase chain reaction) is commonly used to amplify small amounts of a target DNA. Typically 25-35 cycles of amplification are used. The product of the PCR is then detected to infer the amount of the starting nucleic acid target. Since PCR can amplify the starting amount of target DNA by many orders of magnitude, the detection technology needs to be able to accommodate this capability. Unfortunately, many detection technologies only have the ability to linearly detect amounts of DNA over a range of between one and three orders of magnitude. Dudley et al., Proc. Natl. Acad. Sci. USA 99:7554-59 (2002); Kuhn et al, Genome Res. 14:2347-56 (2004); Yang et al., Genome Res. 11:1888-98 (2001); Spiro et al., Appl. Environ. Microbiol., 66:4258-65 (2000).

One method to expand the effective dynamic range of an end-point PCR reaction is to remove a portion of the reaction volume at various time intervals during the amplification process and measure the amount of the amplification product. Alternatively, multiple parallel PCRs can be run and stopped at various time intervals to produce reactions that have been amplified through a different number of cycles. Both of these approaches result in expanding the effective dynamic range of PCR reactions, but the approaches are labor intensive and inconvenient.

There are other approaches to make end point PCR more quantitative. In one approach, nucleic acid “competitors,” whose amplification competes with the intended target, are added to PCR reactions in known concentrations. When the intensity of the amplified target matches that of one of the competitor targets, the starting target amount is equivalent to the competitor. Competitive PCR can be performed with different amounts of a single competitor in different PCR reactions, or distinguishable competitors can be amplified in the same reaction. Vener et al., J. Clin. Microbiol. 36:1864-70 (1998), describe a single tube assay that uses four competitors which differ in size and are analyzed on an automated fluorescence-based sequencer. They point out that the sequencer instrument has only a dynamic range of 100-1000 fold. By diluting their samples five fold and doing two separate measurements, they are able to obtain an effective dynamic range covering the interval between 40 and 500,000 copies of HIV RNA. This approach is also the subject of U.S. Pat. No. 5,837,501.

Real-time PCR is a widely used method that circumvents many of the problems of limited dynamic range that are seen with end point PCR. Real-time PCR provides a measurement of the amount of the nucleic acid amplicon at each cycle. Thus, targets in widely differing amounts can be quantified under the same reaction conditions. An example of a common application of real-time PCR is in the quantification of RNA species, such as mRNA transcripts. This quantification is achieved using an initial reverse transaction step to first convert the RNA to a complementary DNA module (cDNA). Real-time PCR can then be used to amplify and quantify the cDNA. Thus the process of RNA detection using real-time PCR is referred to as reverse transcription-PCR, or RT-PCR. Typically in real-time RT-PCR, the amplified products are normalized against an internal control such as a transcript that is relatively consistently expressed between tissue types, cell types, or under different conditions. In addition, for accurate quantitation of a target RNA, the control transcript and target RNA should be available in similar relative abundance so that a more abundant control transcript does not out-compete a less abundant target for reagents. However, most endogenous control transcripts, such as p-actin, glyceraldehyde-3-phosphate dehydrogenase (GAPDH), or 18s rRNA, are abundantly expressed. Moreover, the amount of the target RNA is often unknown and can be quite variable. Therefore, there is often a discrepancy between the abundance of the target RNA to be amplified, and the endogenous control transcript. This discrepancy can create large errors in quantification.

A number of solutions have been proposed to attenuate amplification or detection of overabundant targets and increase the accuracy of the measurement. One approach is to attenuate the efficiency of the amplification reaction of the more abundant target. U.S. Pat. No. 6,057,134 describes the strategy of mixing Competimers™, nonfunctional PCR primers that are blocked at their 3′ end, with normal, fully functional primers to attenuate amplification. For example, in a PCR reaction in which rRNA and an mRNA are being amplified in the same tube, the rRNA primers might be mixed with Competimers™ such that the amount of amplified rRNA cDNA is comparable to the amount amplified from the mRNA cDNA. However, in cases in which the amount of the abundant target is unknown, multiple reactions would be used to titrate the attenuation.

In another approach, U.S. Pat. No. 7,101,663 discloses a method of balancing multiplex PCR reactions using two sets of primers to amplify two different targets. A further approach is to target two different regions of a target with two different primer sets which will amplify the target with differing efficiencies. This is the subject of U.S. Pat. No. 5,858,732. Such coamplification techniques which employ different target and control sequences amplified by different PCR primer pairs can reduce the accuracy of the method by making it difficult to balance amplification efficiencies.

Although the methods described above provide mechanisms for modulating the dynamic range of an amplification or detection, they require multiple manipulations of a target sequence in the form of serial dilutions, prior measurement of the target, and/or addition of exogenous competing reaction components, in order to alter the dynamic range of a nucleic acid assay. Where it is necessary to screen a large number of samples, such as with high throughput screening, such methods are labor intensive and cumbersome. Further, many amplification reactions may be required to obtain an accurate and reliable measurement with such methods.

There is a need for improved multiplex nucleic acid detection and quantification methods.

### SUMMARY OF THE INVENTION

This invention relates to multiplex nucleic acid detection and quantification methods. In one aspect, a method of the invention comprises determining the amount of a target nucleic acid in a sample. The invention allows multiple distinguishable amplicons to be generated from a single target nucleic acid sequence. Each distinguishable amplicon comprises a distinguishing tag, which allows each amplicon to be differentiated from other amplicons. Accordingly, multiple amplification reactions of multiple distinguishable amplicons can be performed in a single reaction volume.

In one embodiment, the method of the invention comprises: (a) obtaining multiple distinguishable amplicons of the target nucleic acid sequence, each comprising a distinguishing tag and a target portion, wherein each target portion is complementary to an identical target nucleic acid subsequence or its complement; (b) amplifying the amplicons in a single reaction volume; and (c) detecting nucleic acids amplified from at least two distinguishable amplicons. In a different embodiment, the multiple distinguishable amplicons comprise a target portion that is identical in sequence and length in each distinguishable amplicon

In one aspect of the invention, the method further comprises comparing the amount of the nucleic acids detected, thereby increasing target measurement reliability. In another aspect, the method further comprises detecting different amounts of at least two distinguishable amplicons, thereby increasing the dynamic range of the method of determining the amount of the target.

In another aspect, the method includes obtaining the multiple distinguishable amplicons in different amounts, thereby increasing the dynamic range of the method of determining the amount of the target. Additional methods include amplifying the distinguishable amplicons at different amplification efficiencies in certain aspects, thereby increasing the dynamic range of the method of determining the amount of the target in an aspect of the invention. And finally, a further aspect of the invention comprises differentially detecting the nucleic acid target to increase the dynamic range of the method of determining the amount of the target in some methods.

In any of the methods above, the following additional features are provided. In one embodiment, different amounts of each of the multiple distinguishable amplicons are obtained by using different concentrations of primers to the target nucleic acid to produce different amounts of distinguishable amplicons. In another embodiment, different amounts of multiple distinguishable amplicons are obtained using different concentrations of competimers to the target nucleic acid. In another embodiment, the method uses primers that differentially bind to the target nucleic acids to produce different amounts of distinguishable amplicons. In a further embodiment, the method comprises adding oligonucleotides that compete with the target nucleic acid for binding to primers of the target.

In additional embodiments, the invention further comprises: (a) contacting the sample containing the target nucleic acid sequence with multiple distinguishable primers under hybridization conditions, wherein the multiple distinguishable primers comprise: (i) a target binding sequence; and (ii) a distinguishing tag; and (b) producing multiple distinguishable amplicons in a primer-dependent enzymatic reaction. In another embodiment, a method further includes: (a) contacting the sample containing the target nucleic acid sequence with multiple distinguishable probes under hybridization conditions, wherein the multiple distinguishable probes comprise: (i) a target binding sequence; and (ii) a distinguishing tag; and (b) adding a modifying agent to differentiate non-hybridized and hybridized probes.

In some embodiments, the multiple distinguishable amplicons are amplified at different amplification efficiencies to obtain different amounts of the multiple distinguishable amplicons. In one embodiment, the amplification efficiency is attenuated by using different concentrations of amplification primers to at least two distinguishable amplicons. In another embodiment, the amplification efficiency of different amplicons is attenuated by adding competimers to at least one amplicon. In yet another embodiment, the amplification efficiency is altered by using amplification primers that bind with different binding efficiencies to different distinguishable amplicons. In a further embodiment, the amplification efficiency is altered by adding oligonucleotides that compete with distinguishable amplicons for binding with amplification primers.

In other embodiments of the invention, nucleic acids amplified from the multiple distinguishable amplicons are differentially detected, which results in expansion of the dynamic range of the nucleic acid quantification method. In one embodiment, the nucleic acids are detected by probes that bind with different binding efficiencies and/or by competitive hybridization.

In an additional embodiment, the method comprises: (a) obtaining multiple distinguishable sequencons of the target nucleic acid sequence in a single reaction volume, each comprising a distinguishing tag and a target portion, wherein the target portions are complementary to an identical target nucleic acid subsequence; and (b) detecting different amounts of at least two of the sequencons, thereby increasing the dynamic range of the method of determining the amount of the target nucleic acid. As used herein, the term “sequencon” corresponds to “amplicon,” except that amplification of the sequencon is optional. And in a further embodiment, the invention provides a method comprising: (a) obtaining multiple distinguishable amplicons of the target nucleic acid sequence, each comprising a zip code and a target portion, wherein the target portions are complementary to an identical or overlapping target nucleic acid subsequence; (b) amplifying the amplicons in a single reaction volume; and (c) detecting nucleic acids amplified from each of the amplicons.

In additional embodiments, the invention provides a kit for detecting a target nucleic acid sequence in a sample comprising multiple distinguishing tags, wherein at least two of the distinguishing tags comprise: (a) a target binding sequence that is complementary to an identical or overlapping portion of the target nucleic acid sequence; and (b) a zip code that uniquely identifies the distinguishing tag. In certain aspects, at least two of the distinguishing tags further comprise a primer binding site, and/or a distinguishable primer binding site. In other aspects, the kits further comprise multiple amplification primer sets, wherein at least one of the primers in each of the primer sets comprises a sequence that is identical to or complementary to a portion of at least one distinguishing tag. The kits optionally also comprise at least two probes complementary to a portion of at least two distinguishing tags.

Other embodiments of the invention are discussed throughout this application. Other objects, features, and advantages of the present invention will become apparent from the following detailed description. Any embodiment discussed with respect to one aspect of the invention applies to other aspects of the invention as well and vice versa. The embodiments in the Example section are understood to be embodiments of the invention that are applicable to all aspects of the invention.

It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a schematic diagram illustrating an embodiment of the method of the invention. In this embodiment, a target RNA sequence binds to two different reverse transcription (RT) primers each comprising a target binding site complementary to the target sequence, a zip code, and an amplification primer binding site (PBS). Distinguishable amplicons are synthesized from the primers in the RT reaction. The amplicons are then amplified by PCR, and the amplified products are detected, in this case, using a probe containing a quencher molecule (Q) and fluorescent dye molecule (F), such as a TaqMan™ probe.

FIG. 2 is a graph illustrating an effect of RT primer dilution on the dynamic range of a nucleic acid quantification method. Different amounts of distinguishable amplicons to the same target sequence were obtained using different concentrations of different RT primers in a reverse transcription reaction. The amplicons were then amplified by PCR. The threshold cycle (Ct value) for each of the reactions is plotted on the Y-axis, and the starting copy number of the target sequence (here, miRNA let 7a) is plotted on the X-axis. Ct values for Amplicon #1 (made using undiluted primers) are indicated with circles “”. Ct values for Amplicon #2 (made using diluted primers) are indicated with squares“▪”. The data show a 1000-fold attenuation using diluted RT primers to create distinguishable amplicons in this experiment.

FIG. 3 is a graph illustrating an effect of competimers on the dynamic range of a nucleic acid quantification method. Two distinguishable amplicons to the same target sequence were obtained using a reverse transcription reaction. The amplicons were then amplified by PCR in the presence of competimers to Amplicon #2 only. The threshold cycle (Ct value) for each of the reactions is plotted on the Y-axis, and the starting copy number of the target sequence (here, miRNA let 7a) is plotted on the X-axis. Ct values for Amplicon #1 are indicated with circles “”. Ct values for Amplicon #2 are indicated with squares “▪”. The data show that a 100-fold attenuation was achieved.

FIG. 4 is a graph illustrating an effect of using amplification primers with different melting temperatures on the dynamic range of a nucleic acid quantification method. Two distinguishable amplicons to the same target sequence were obtained using a reverse transcription reaction. The amplicons were then amplified using amplification primers of different lengths. The threshold cycle (Ct value) for each of the reactions is plotted on the Y-axis, while the starting copy number of the target sequence (here, miRNA let 7a) is plotted on the X-axis. Ct values for Amplicon #1 (obtained using normal length primers) are indicated with circles “”. Ct values for Amplicon #2 (obtained using 9-mer primers) are indicated with triangles “▴”. Ct values for Amplicon #2 (obtained using 8-mer primers) are indicated with squares “▪”. The graph illustrates that signal attenuation of greater than 10,000-fold is achieved by using primers with different melting temperatures in the amplification reaction.

FIG. 5a depicts a competitive hybridization scheme to attenuate detection of an amplified nucleic acid. B is a biotinylated target molecule. AS indicates an anti-sense molecule. S indicates a sense molecule. In this scheme, the non-biotinylated oligonucleotide competes with the biotinylated target for binding with the detection sequence on the detection probe.

FIG. 5b depicts an alternative competitive hybridization scheme to attenuate detection of an amplified nucleic acid. B is a biotinylated target molecule. AS indicates an anti-sense molecule. S indicates a sense molecule. In this scheme, the non-biotinylated oligonucleotide that is complementary to the biotinylated target molecule competes with the detection probe for binding to the target nucleic acid.

FIG. 6a is a graph illustrating the effects of competitive hybridization on the dynamic range of a detection assay for the target sequence capture oligo-1 (CO-1) (SEQ ID NO:11). Biotinylated target sequences were detected using the Luminex apparatus. Net Median Fluorescence Index (MFI) of detected targets is plotted on the Y-axis, while amounts of the target sequence are plotted on the X-axis. The graph illustrates that, in this case, the competitive hybridization scheme set out in FIG. 5b produces a more linear (dose dependent) response to increasing amounts of target than the scheme set out in FIG. 5a.

FIG. 6b is a graph illustrating the effects of competitive hybridization on the dynamic range of a detection assay for the target sequence capture oligo-2 (CO-2) (SEQ ID NO:12). As in the case of FIG. 6a, Net Median Fluorescence Index (MFI) of detected targets is plotted on the Y-axis, while amounts of the target sequence are plotted on the X-axis. The graph illustrates that, in this case, the competitive hybridization scheme set out in FIG. 5b produces a more linear (dose dependent) response to increasing amounts of target than the scheme set out in FIG. 5a.

FIG. 7 is a graph depicting dynamic range expansion of a detection assay using the competitive hybridization scheme illustrated in FIG. 5b. Detection results from assays conducted without adding competing oligonucleotides are indicated with triangles “▴” symbol. Detection results using competing oligonucleodies are indicated with squares “▪” symbol. The graph illustrates that the competitive hybridization scheme in FIG. 5b attenuates the target detection such that target amounts normally outside the dynamic range of the detection method are now within the dynamic range.

FIG. 8 is a graph depicting attentuation by competitive hybridization using CO-2 (SEQ ID NO:12). The amount of biotin complement oligonucleotide in pmol is plotted along the X-axis, and the MFI is plotted on the Y-axis. Each line represents a different amount of non-biotinylated oligonucleotide, resulting in ratios of biotin:non-biotin oligo ranging from 1:25 to 1:4000.

FIG. 9 is a schematic diagram of various embodiments of the methods of the invention, depicting differential and non-differential aspects of the methods (left to right). As is apparent, multiple distinguishable amplicons may be differentially obtained (a_{d}), amplified (b_{d}), and/or detected (c_{d}), or they may be obtained, amplified, and/or detected without differentiating between the amplicons (noted as a, b, and c, respectively).

### DETAILED DESCRIPTION

This application provides multiplex methods of determining the amount of a target nucleic acid in a sample. In accordance with one aspect of the invention, multiple distinguishable amplicons are generated from a target nucleic acid sequence. Each distinguishable amplicon comprises a distinguishing tag, which allows an amplicon to be differentiated from other amplicons and a target portion. Accordingly, multiple amplification reactions are performed in a single reaction volume and multiple measurements of a single target can be obtained. In addition, the dynamic range of the assay can be expanded when the multiple distinguishable amplicons are differentially obtained, amplified, and/or detected. Thus, the methods can increase the precision, reliability and/or dynamic range of detecting a target nucleic acid in a sample.

To assist in understanding the present invention, certain terms are first defined. Additional definitions are provided throughout the application.

As used herein, the terms “complementary” or “complementarity” are used in reference to polynucleotides (i.e., a sequence of nucleotides) related by the base-pairing rules. For example, the sequence “5′-A-G-T-3′” is complementary to the sequence “3′-T-C-A-5′.” The nucleic acid sequences comprise natural nucleotides (including their hydrogen bonding bases A, C, G, T, or U) and modified nucleotides or bases. Complementarity may be “partial,” in which less than all of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. As used herein, a hybridizing nucleic acid sequence is “substantially complementary” when it is at least 90% identical and/or includes no more than one non-Watson-Crick base pairing interaction to a reference sequence in the hybridizing portion of the sequences.

As used herein, a “distinguishing tag” is a sequence that allows an amplicon to be separately detected or amplified. A distinguishing tag comprises a nucleic acid sequence that is different from the sequence of the target nucleic acid and uniquely identifies the tag from other distinguishing tags. In some embodiments, the distinguishing tag comprises a “zip code.” A zip code is a unique differentiating sequence, which facilitates selective detection of the distinguishable amplicon. In certain embodiments, a zip code is complementary to a capture tag on a Luminex bead, for example. A zip code may be a distinguishable probe. In certain embodiments, the distinguishing tag comprises a distinguishable primer binding site to allow selective amplification of the distinguishable amplicon. A distinguishing tag may comprise both detection and amplification portions, for example comprising a zip code and a primer binding site. In additional embodiments, a distinguishing tag comprises multiple primer binding sites, such as nested primer binding sites, to allow selective amplification and/or detection of the nucleic acid sequence.

The terms “hybridize,” “hybridization,” and their cognates are used herein to refer to the pairing of complementary nucleic acids or bases. Hybridization and the strength of hybridization (i.e., the strength of the association between the nucleic acids) is influenced by such factors as the degree of complementarity between the nucleic acids, stringency of the hybridization conditions involved, the melting temperature (Tm) of the formed hybrid, and the G:C ratio within the nucleic acids. A hybridizing sequence is at least 75%, 80%, 85%, 90%, 92%, 94%, 95%, 96%, 97%, 98%, 99%, or 100% identical and is matched according to the base pairing rules. It may contain natural and/or modified nucleotides and bases.

The term “label” refers to any molecule that can be detected. In certain embodiments, a label can be a moiety that produces a signal or that interacts with another moiety to produce a signal. In certain embodiments, a label can interact with another moiety to modify a signal of the other moiety. In certain embodiments, a label can bind to another moiety or complex that produces a signal or that interacts with another moiety to produce a signal. In certain embodiments, the label emits a detectable signal when the probe is bound to a complementary target nucleic acid sequence. In certain embodiments, the label emits a detectable signal when the label is cleaved from the polynucleotide probe. In certain embodiments, the label emits a detectable signal when the label is cleaved from the polynucleotide probe by a 5′ exonuclease reaction.

A “primer” is an oligonucleotide that is capable of binding to a complementary target nucleic acid sequence. In certain embodiments, a primer is used to initiate a nucleic acid extension reaction.

A “probe” is a polynucleotide that is capable of binding to a complementary target nucleic acid sequence. In certain embodiments, a probe is used to detect amplified target nucleic acid sequences. In certain embodiments, the probe incorporates a label.

As used herein, “reagents” for any enzymatic reaction mixture, such as a reverse transcription and PCR reaction mixture, are any compound or composition that is added to the reaction mixture including, without limitation, enzyme(s), nucleotides or analogs thereof, primers and primer sets, buffers, salts, and co-factors. As used herein, unless expressed otherwise, “reaction mixture” includes all necessary compounds and/or compositions necessary to perform that enzymatic reaction, even if those compounds or compositions are not expressly indicated.

A “target” nucleic acid sequence is a nucleic acid molecule, such as DNA, cDNA, genomic DNA, mitochondrial DNA, RNA, mRNA, miRNA, siRNA, piwi-interacting RNA, rRNA, tRNA, snRNA, viral RNA, and fragments and segments thereof. A target sequence can be single-stranded, double-stranded, continuous, or fragmented, so long as a distinguishing tag can hybridize and amplify or detect a subsequence of the target. The target may be a gene, a gene fragment, or an extra-chromosomal nucleic acid sequence, for example. As used herein, a “subsequence” is a portion of a sequence, such as a target nucleic acid sequence, that is contained within the longer sequence.

### I. Methods to Determine the Amount of a Target Nucleic Acid

In one embodiment, the method of the invention comprises: (a) obtaining multiple distinguishable amplicons of the target nucleic acid sequence, each comprising a distinguishing tag and a target portion; (b) amplifying the amplicons in a single reaction volume; and (c) detecting nucleic acids amplified from at least two of the amplicons. In another embodiment, the method of the invention comprises: (a) obtaining multiple distinguishable amplicons of the target nucleic acid sequence in a single reaction volume, each comprising a distinguishing tag and a target portion; and (b) differentially detecting nucleic acids from at least two of the amplicons. The target portions of the distinguishable amplicons are identical in sequence and length in two or more distinguishable amplicons in certain aspects of the invention, and the target portions are complementary to an identical portion of the target nucleic acid sequence (a “target nucleic acid subsequence”) in two or more distinguishable amplicons in certain aspects of the invention.

In another embodiment, the method comprises: (a) obtaining multiple distinguishable sequencons of the target nucleic acid sequence in a single reaction volume, each comprising a distinguishing tag and a target portion, wherein the target portions are complementary to an identical target nucleic acid subsequence; and (b) detecting different amounts of at least two of the sequencons, thereby increasing the dynamic range of the method of determining the amount of the target nucleic acid. A “sequencon” is a term used to make clear that in this embodiment of the invention, the same nucleic acid obtained in step (a) is not destined for amplification. As stated previously, “sequencon” corresponds to “amplicon.” To the extent that characteristics, properties, and aspects of amplicons are provided in this application, the characteristics apply to a sequencon as well, with the proviso that a sequencon is optionally amplified.

In certain embodiments the sequencon is differentially obtained. In other embodiments, the sequencon is differentially detected. In some embodiments, it is amplified, including differentially amplified. The target portions of the distinguishable sequencons are identical in sequence and length in two or more distinguishable sequencons in certain aspects of the invention, and the target portions are complementary to an identical or overlapping portion of the target nucleic acid sequence (a “target nucleic acid subsequence”) in two or more distinguishable sequencons in certain aspects of the invention. In embodiments in which the distinguishable nucleic acids are obtained and detected by non-amplification methods, the sequences referred to herein as “sequencons,” are detected in different amounts. In some aspects of this embodiment, the sequencons are differentially detected with signal amplification.

In a further embodiment of the invention, any of the methods above may obtain multiple distinguishable amplicons, each comprising a zip code and a target portion that is complementary to an identical or overlapping target subsequence or its complement.

In certain embodiments, the methods further comprise comparing the amount of nucleic acids amplified from at least two distinguishable amplicons. In various aspects of the invention, multiple measurements are averaged or otherwise analyzed to increase the accuracy, precision, and/or reliability of the method or of the measurement. A matching standard curve is generated to provide a reference for the extraction of quantitative information from the sample measurements in embodiments of the invention. In exemplary aspects, the standard curve spans the range of known inputs of target nucleic acid sequence. If the multiple distinguishable amplicons are produced during the amplification step, then the standard curve is generated from separate reactions that contain defined inputs of the target nucleic acid(s) amplified with the same reagents and under the same conditions used to assay the sample of consideration. Signals produced from a known amount of target can be compared with signals from unknown target quantities following the detection step to reveal the target concentration, provided the sample signal derived from at least one of the multiple distinguishable amplicon lies within the dynamic range window that the instrument can accurately report. The number of distinguishable amplicons that are required for the assay is dependent on the level of dynamic range expansion that is required; e.g., three differentially attenuated amplicons can provide greater range expansion than two. Moreover, each distinguishable amplicon is associated with a separate standard curve that empirically links assay the signal intensities for each with the underlying concentration of the input target sequence as generated in the appropriately multiplexed reaction. For example, the unattenuated amplification reaction can be performed in the presence of a range of known inputs that far surpasses the known dynamic range of the instrument and extends to the limits of detection of the most attenuated amplicon that can be distinguished in the multiplexed reaction. The unattenuated reaction only reports dose-dependent data, however, within the nominal instrument dynamic range. At the point wherein the unattenuated reaction becomes saturated in its signal response and further increases in the target nucleic acid abundance are no longer dependent on the concentration of said target, sufficient attenuation in the first attenuated amplification reaction is necessary to report a more muted signal that again falls within the responsive instrument dynamic range. This strategy can continue in stepwise increments that roughly correspond to windows of quantification whose breadth spans values defined by the underlying instrument dynamic range. In this way, the dynamic range of the assay is expanded in a manner that is dependent on the number of distinguishable amplicons that are employed, and the levels of attenuation that can be achieved with each. In this example, data for any given input of all amplicon standard curves are produced within a single reaction tube by virtue of the multiplexed assay design that reveals unique signals for each of the multiple distinguishable amplicons.

If the multiple distinguishable amplicons are disproportionately produced during the signal reporting step, rather than the amplification reaction, then a standard curve is generated from separate reactions that contain defined inputs of the target nucleic acid(s) detected with the same reagents and under the same conditions used to assay the sample of consideration. As above, signals produced from a known amount of target can be compared with signals from unknown target quantities to reveal the target concentration, provided the sample signal derived from at least one of the multiple distinguishable amplicons lies within the dynamic range window that the instrument can accurately report. In preferred embodiments, the amount of target nucleic acid sequence in the sample is quantified based on the amount of amplified nucleic acid and/or the amount of the distinguishable amplicon detected using methods known in the art.

In one example using the Luminex bead array platform to detect nucleic acids, the dynamic range of nucleic acid quantification is expanded by staggering multiple windows of dynamic range that overlap within the approximately two log dynamic range limitation of the Luminex assay. This enables continuous coverage of signal responses across the broader range. When a high sample within the range provides high signals that can be extrapolated to a standard curve and the same sample returns a low signal from a low efficiency reaction that is still quantifiable, the amount of target may be determined by more than one independent calculation. This provides more data from fewer samples.

Accordingly, the methods of the invention provide a convenient and efficient manner of obtaining multiple duplicate assays of a single target nucleic acid in a single reaction vessel. Multiple measurements within the linear range of the assay allow increased reliability of the measurement, since each distinguishable amplicon is an independent determination of the concentration of the target. If distinguishable amplicons to the same target subsequence are detected at more than one point within the linear range of the assay, for example, the accuracy, precision, and/or reliability of the measurement of the amount of target nucleic acid sequence can be increased.

A. Distinguishable Amplicons

In one embodiment, multiple distinguishable amplicons are obtained using primer-dependent polymerization reactions, such as reverse transcriptase or DNA-polymerase dependent reactions. In a primer-dependent polymerization reaction, distinguishable amplicons may be obtained with a distinguishing tag that includes a primer binding portion and a target binding portion. In this embodiment, once a distinguishable amplicon is made using the primer, the distinguishable amplicon also comprises a distinguishing tag. In addition, in certain embodiments the distinguishing tags comprise different primer binding sites to allow the multiple distinguishable amplicons to be separately amplified.

All disclosure relating to amplicons, as defined herein, equally defines “sequencon,” as used in specific embodiments of the methods of the invention. Unlike an amplicon, a sequencon is optionally amplified or to be amplified.

In certain aspects, multiple distinguishable amplicons are obtained with reverse transcriptase reactions, wherein a target RNA sequence in a sample is hybridized to multiple distinguishable primers, and the amplicons are produced as cDNA using reverse transcriptase. Alternatively, the distinguishable amplicons are produced from a target DNA sequence using a nucleic acid polymerization method, which may amplify the target, such as by using a DNA polymerase and distinguishable primers as distinguishing tags.

In aspects of the invention, the multiple distinguishable amplicons are obtained by amplification reaction. Suitable nucleic acid polymerization and amplification techniques include reverse transcription, PCR, transcription-mediated amplification (TMA), nucleic acid sequence-base amplification (NASBA), rolling circle amplification, whole genome amplification (WGA) including via phi29 polymerase or PCR-type approaches, in vitro transcription, RNA (Eberwine) amplification, invader technology, ligase chain reaction, strand displacement amplification, multiplex ligatable probe amplification, and other methods that are known to persons skilled in the art.

In some aspects the polymerase is a thermostable polymerase. Exemplary thermostable polymerases include, but are not limited to, Thermus thermophilus HB8 (see e.g., U.S. Pat. No. 5,789,224 and U.S. Publication No. 20030194726); mutant Thermus oshimai; Thermus scotoductus; Thermus thermophilus 1B21; Thermus thermophilus GK24; Thermus aquaticus polymerase (AmpliTaqB FS or Taq (G46D; F667Y) (see e.g., U.S. Pat. No. 5,614,365), Taq (G46D; F667Y; E6811), and Taq (G46D; F667Y; T664N; R660G); Pyrococcus furiosus polymerase; Thermococcus gorgonarius polymerase; Pyrococcus species GB-D polymerase; Thermococcus sp. (strain 9″N-7) polymerase; Bacillus stearothermophilus polymerase; Tsp polymerase; ThermalAce™ polymerase (Invitrogen); Thermusflavus polymerase; Thermus litoralis polymerase and mutants or variants thereof.

Exemplary non-thermostable polymerases include, but are not limited to DNA polymerase I; mutant DNA polymerase 1, including, but not limited to, Klenow fragment and Klenow fragment (3′ to 5′ exonuclease minus); T3, T4, T5, T7, or phi29 DNA polymerase; and mutants or variants thereof.

In further aspects of the invention, multiple distinguishable amplicons are obtained using a non-amplification method, such as methods relying on hybridization and/or ligation to obtain multiple distinguishable amplicons. Exemplary methods include oligonucleotide ligation (OLA) methods and methods that allow distinguishable probe that hybridizes to the target nucleic acid sequence to be separated from unbound probe. In some aspects, a probe may affect the efficiency of an enzymatic reaction, such as nucleic acid polymerization or ligation. In the case of OLA, this could be a modification that reduces the efficiency of ligation for some probe pairs that bind to the target, but not others. Thus, differential detection of multiple distinguishable amplicons may be mediated by a differential effect on an enzyme-mediated reaction, on hybridization, or on amplification, for example.

As an example, HARP-like probes, as disclosed in U.S. Publication No. 2006/0078894 (incorporated herein by reference) may be used to obtain multiple distinguishable amplicons. In such methods, after hybridization between a probe and the targeted nucleic acid, the probe is modified to distinguish hybridized probe from unhybridized probe. Thereafter, the probe may be amplified and/or detected.

In the HARP system, the probe comprises a probe inactivating region, which contains nucleotides that can distinguish hybridized from non-hybridized HARP probe. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify unhybridized HARP probe such that it cannot be amplified. In some embodiments the agent can also be used to distinguish a HARP probe that is hybridized to a completely complementary target nucleic acid sequence from a HARP probe hybridized to a related target containing one or more basepair mismatches with the HARP probe, for the purpose of distinguishing between related target sequences. In an embodiment the HARP probe includes nucleotides that can be cleaved by a cleaving agent when they are in their unhybridized, single-stranded state, and which are resistant to cleavage when they are hybridized to their target nucleic acid. The HARP probes may also contain nucleotides that cannot be cleaved (non-cleavable portion), regardless of whether they are single-stranded or double-stranded. An example is a HARP probe composed of DNA and RNA, where the cleaving agent is a ribonuclease, such as RNase A, RNase T1, or RNase 1, that cleaves single stranded RNA; the HARP probe will be susceptible to cleavage in the RNA portion of the molecule when it is not hybridized to a complementary sequence.

In an additional embodiment of the method, a probe ligation reaction obtains multiple distinguishable amplicons. In a Multiplex Ligation-dependent Probe Amplification (MLPA) technique (Schooten et al., Nucleic Acids Research 30:e57 (2002)) pairs of probes which hybridize immediately adjacent to each other on the target nucleic acid are ligated to each other only in the presence of the target nucleic acid. In some aspects, MLPA probes have flanking PCR primer binding sites as well as a zip code, which allows size discrimination of different pairs of MLPA probes. MLPA probes can only be amplified if they have been ligated. Thus, MLPA probes do not have to be washed away from the template before an amplification reaction. Schooten describes an assay with 40 different MLPA probe pairs which upon ligation and PCR amplification can be resolved by size. The assay can be used to identify SNPs, measure gene deletions or duplications, etc.

Certain methods of the invention can be used to expand the dynamic range of a MLPA probe targeted at a specific gene. For example, three different MLPA probes are designed that hybridize to the same target sequence. Each MLPA probe has a different zip code which allows the amplification products of each MLPA probe to be distinguished. Each MLPA probe also has unique PCR primer binding sites allowing each MLPA probe to be amplified with a differing efficiency. Competimers could be used to decrease the amplification efficiencies of two of the MLPA probe amplification reactions, thus increasing the effective dynamic range of the assay.

In various embodiments of the methods, the multiple distinguishable amplicons comprise a target portion, which is identical in sequence and length in at least two distinguishable amplicons in some methods of the invention. The target portions of at least two amplicons comprise a common target subsequence. Target portions can be overlapping: target portions that comprise the same target subsequence or its complementary sequence, but differ at the 5′ and/or 3′ end of the subsequence are useful in the methods of this invention. For example, the target portions of at least two distinguishable amplicons may overlap, but differ by less than or equal to 0, 1, 2, 3, 4, 5, 7, 10, 15, or 20 nucleotides at their 5′ and/or 3′ ends. The target portions may be complementary to or they may hybridize to an identical target subsequence or its complement in various methods. When obtained with primers containing a mismatch, for example, the target portion of the multiple distinguishable amplicons will not be totally complementary to each other; the hybridizing portions of the distinguishing tag may include less than or equal to 1, 2, 3, 4, 5, 7, 10, 15, or 20 mismatches, depending on the hybridizing length.

B. Methods to Amplify Multiple Distinguishable Amplicons in a Single Reaction Volume

In some embodiments of the invention, the multiple distinguishable amplicons are amplified in a single reaction volume. As described above, the distinguishable amplicons may be differentially amplified, or at least two distinguishable amplicons may be amplified at the same amplification efficiency, at about the same amplification efficiency, or at a similar amplification efficiency. As used here (and throughout this application) the term “about” is used to indicate that a value within two times the standard deviation of error for the device or method employed to determine the value. A value is the “same” when it is within the standard deviation of error for the device and/or method.

Amplification of more than one amplicon in a single reaction volume, for example with multiplex PCR, enables simultaneous amplification of at least one target of interest in one reaction by using more than one pair of primers. Multiplex amplification reactions are applied in many areas of nucleic acid testing, including analyses of deletions, mutations, polymorphisms, quantitative assays, and reverse transcription PCR. Multiplex PCR has research and diagnostic uses, including but not limited to genotyping applications where simultaneous analysis of multiple markers is required, detection of pathogens or genetically modified organisms, or microsatellite analyses. Multiplex assays have been used to simultaneously detect and identify different target nucleic acid sequences. In various embodiments of the invention, multiplex PCR enables multiple measurements of a target sequence or subsequence and/or it enables multiple measurements to expand the dynamic range of detection of the target.

Further, the distinguishable amplicons have the same or similar amplification efficiency in some circumstances, and the amplicons have different amplification efficiencies in other methods described herein. Amplification efficiency is impacted by intrinsic and extrinsic factors, which may affect the melting, hybridization, and/or polymerization steps of an amplification reaction.

In certain instances PCR is used. In an amplifying step, multiple distinguishable amplicons may be amplified using an amplification primer set comprising at least one amplification primer that uniquely binds the distinguishing tag of each distinguishable amplicon. In one embodiment, the amplification primer binds a unique primer binding site in the distinguishing tag. In another embodiment, the amplification primer binds to the zip code in the distinguishing tag.

Suitable amplification techniques include reverse transcription, PCR, transcription-mediated amplification (TMA), nucleic acid sequence-base amplification (NASBA), rolling circle amplification, whole genome amplification (WGA) including via phi29 polymerase or PCR-type approaches, in vitro transcription, RNA (Eberwine) amplification, invader technology (Third Wave), ligase chain reaction, strand displacement amplification, multiplex ligatable probe amplification, and other methods that are known to persons skilled in the art.

C. Detection of Nucleic Acids

Many methods of detecting nucleic acids are contemplated. In some embodiments of the invention, the distinguishable amplicons and/or the nucleic acids amplified therefrom are differentially detected, for example at different efficiencies or with different signal amplification or strength. In certain embodiments, the distinguishable amplicons are separately identified in the detection step, but not differentially detected.

In certain embodiments, a probe may include Watson-Crick bases or modified bases. Modified bases include, but are not limited to, the AEGIS bases (from Eragen Biosciences), which have been described, e.g., in U.S. Pat. Nos. 5,432,272; 5,965,364; and 6,001,983. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a further aspect, oligonucleotide probes present in a multiplex amplification are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TaqMan™) probes (see above and also U.S. Pat. No. 5,538,848), stem-loop molecular beacons (see, e.g., U.S. Pat. Nos. 6,103,476 and 5,925,517), stemless or linear beacons (see, e.g., WO 9921881, U.S. Pat. Nos. 6,485,901 and 6,649,349), peptide nucleic acid (PNA) Molecular Beacons (see, e.g., U.S. Pat. Nos. 6,355,421 and 6,593,091), linear PNA beacons (see, e.g. U.S. Pat. No. 6,329,144), non-FRET probes (see, e.g., U.S. Pat. No. 6,150,097), Sunrise™/AmplifluorB™probes (see, e.g., U.S. Pat. No. 6,548,250), stem-loop and duplex Scorpion™ probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse™ probe (Epoch Biosciences), hairpin probes (see, e.g., U.S. Pat. No. 6,596,490), PNA light-up probes, antiprimer quench probes (Li et al., Clin. Chem. 53:624-633 (2006)), self-assembled nanoparticle probes, and ferrocene-modified probes described, for example, in U.S. Pat. No. 6,485,901.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more different labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

Nucleic acids, including the multiple distinguishable amplicons of the invention, can be detected by direct or indirect methods. In a direct detection method, the distinguishable amplicons are detected by a detectable label that is linked to a nucleic acid molecule. In such methods, the amplicons may be labeled prior to binding to the probe. Therefore, binding is detected by screening for the labeled amplicon that is bound to the probe. The probe is optionally linked to a bead (such as in a bead array) or to a solid support (such as in a planar array).

In certain embodiments of the invention, amplified nucleic acids are detected by direct binding with a labeled probe, and the probe is subsequently detected. In one embodiment of the invention, the amplified nucleic acids are detected using FlexMAP Microspheres (Luminex) conjugated with probes to capture the desired amplified nucleic acids. Some methods may involve detection with polynucleotide probes modified with fluorescent labels or branched DNA (bDNA) detection, for example.

In other embodiments of the invention, amplified nucleic acids are detected by indirect detection methods. In such an embodiment, a biotinylated probe is combined with a stretavidin-conjugated dye to detect the bound nucleic acid. The streptavidin molecule binds the biotin label on the amplicon, and the bound amplicon is detected by detecting the dye molecule attached to the streptavidin molecule. In one embodiment, the streptavidin-conjugated dye molecule comprises Phycolink® Streptavidin R-Phycoerythrin (PROzyme). Other conjugated dye molecules are known to persons skilled in the art.

Labels include, but are not limited to, light-emitting, light-scattering, and light-absorbing compounds which generate or quench a detectable fluorescent, chemiluminescent, or bioluminescent signal (see, e.g., Kricka, 1992 and Garman, 1997). Fluorescent reporter dyes useful as labels include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., WO 9745539), lissamine, phycoerythrin, Cy2, Cy3, Cy3.5, Cy5, Cy5.5, Cy7, FluorX (Amersham), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein. In certain aspects, the fluorescent label is selected from SYBR-Green, 6-carboxyfluorescein (“FAM”), TET, ROX, VICTM, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluor and a quencher fluor is used in some embodiments. It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used. However, because of scattering of radioactive particles and the consequent requirement for widely spaced binding sites, in the context of arrays (such as planar arrays) the use of radioisotopes is a less-preferred embodiment.

In still a further aspect, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)). Labels include those labels that effect the separation or immobilization of a molecule by specific or non-specific capture, for example biotin, digoxigenin, and other haptens.

In other aspects, labeled DNA (or RNA) is prepared by incorporating a nucleotide, such as an NTP or dNTP, conjugated to a detectable label, most preferably a fluorescently labeled dNTP. In alternative embodiments, the DNA or RNA probe can be synthesized in the absence of a detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

In one aspect, a labeled DNA probe is synthesized by incubating a mixture containing 0.5 mM dGTP, dATP, and dCTP plus 0.1 mM dTTP plus fluorescent deoxyribonucleotides (e.g., 0.1 mM Rhodamine 110 UTP (Perkin Elmer) or 0.1 mM Cy3 dUTP (GE Healthcare)) with RNA template and reverse transcriptase (e.g., SuperScript™, Invitrogen Inc.) at 42° C. for 60 min.

### II. Expaning Dynamic Range of the Nucleic Acid Quantification

One advantage of the method of the invention is that the amounts of the distinguishable amplicons can be individually varied in each of the steps of the method, which allows the dynamic range of the nucleic acid quantification method to be expanded. Because each distinguishable amplicon comprises a distinguishing tag that allows each distinguishable amplicon to be differentially amplified or detected, multiple distinguishable amplicons that share a subsequence of a target nucleic acid can be differentially obtained, amplified, and/or detected in a single reaction volume, for example. Thus the method of the invention provides a convenient way to obtain multiple measurements of a target nucleic acid quantity.

A. Differentially Obtaining Distinguishable Amplicons

In some embodiments, the dynamic range of the quantification method of the invention is expanded by using different amounts of distinguishable amplicons in the method.

According to the invention, the different assays for obtaining distinguishable amplicons can be combined with the assays for differential amplification and/or differential detection of the amplicon or amplified nucleic acids. Such embodiments can accommodate broad ranges of target nucleic acid input amounts. Furthermore, distinguishable amplicons to one or more target nucleic acid sequences can be analyzed, as the distinguishing tags allow the distinguishable amplicons to be independently obtained, amplified, and/or detected in a single reaction volume.

1. Primer Concentrations and Competitive Binding

In one embodiment, different amounts of distinguishable amplicons are obtained by contacting a sample containing a target nucleic acid with different dilutions of different distinguishable primers under hybridization conditions and producing the distinguishable amplicons through a primer-based enzymatic reaction. The distinguishable amplicons are then amplified in a single reaction comprising the multiple distinguishable amplicons and amplification primers unique to each distinguishable amplicon. The different amounts of amplified nucleic acids are then detected and quantitated. By diluting the distinguishable primers in a step-wise manner over a large range of concentrations, at least one of the resulting amplified nucleic acid amounts will be detectable within the dynamic range of the detection method, regardless of the amount of the target nucleic acid in the sample. Primers may be diluted by at least or about 5-fold, 10-fold, 25-fold, 50-fold, 75-fold, 100-fold, 200-fold, 300-fold, 400-fold, 500-fold, 750-fold, 1000-fold, 10000-fold, 50000-fold, 100000-fold, (including all values and ranges there between), for example to attenuate the amplification efficiency of a reaction.

In another embodiment, different amounts of distinguishable amplicons are obtained by contacting a sample containing a target nucleic acid with distinguishable primers in the presence of competimers to one or more of the distinguishable primers. The presence of a competimer to a distinguishable primer attenuates the binding of the distinguishable primer, which in turn reduces the amount of distinguishable amplicon produced using the distinguishable primer. In an alternative embodiment, the sample containing the target nucleic acid is contacted with distinguishable primers in the presence of oligonucleotides that compete with the target nucleic acid for binding to one or more distinguishable primers. Portions of WO0218616 describing template-mimic oligos (TMOs) structure and use are herein incorporated by reference. Again, the attenuation of the binding of the distinguishable primer to a target reduces the amount of distinguishable amplicon produced by the distinguishable primer. Competitive binding encompasses trans competition, with a second nucleic acid molecule, and cis competition, in which a single molecule includes a competitively binding sequence. Exemplary cis competition reactions include nucleic acids that form hairpin or loop structures and nucleic acids that have lower single-stranded to double-stranded character.

As used herein, the term “competimer” refers to a nucleic acid probe that hybridizes to the same sequence or an overlapping sequence as a primer but is blocked at the 3′ terminus and thus cannot be extended in primer-dependent enzymatic reactions. Competimers™ are the subject of U.S. Pat. No. 6,057,134, which is incorporated herein by reference.

Competimers™ are modified oligonucleotides which do not have free 3′ hydroxyl groups and which compete with unmodified oligonucleotide primers for binding sites on nucleic acid molecules. Such competition, if it occurs during a primer extension reaction like PCR or reverse transcription decreases the chance of the DNA polymerase finding a primer in place on the template, and thus lowers the efficiency of the reaction. A Competimer™ may be an RNA, DNA, DNA/RNA chimera, or peptide nucleic acid (PNA), for example. A 3′ terminal hydroxyl group of the Competimer™ may be modified by any number of means, for example, 3′ addition of phosphate, biotin, digoxygenin, fluorescein, a dideoxynucleotide, an amine, a thiol, an azo (N₃) group, or fluorine. Oligonucleotides may be synthesized on automated machines from the 3′ to the 5′ direction, with the 3′ base supplied coupled to the controlled pore glass (cpg) synthesis column. Modifications to the 3′ end, such as phosphorylation, biotin, amine, sulfhydryl, and fluorescein are supplied attached to the 3′ hydroxyl on the column as purchased, for example, from Genosys Biotechnologies (800) 234-5362, DNA Technologies (800) 998-3628, Midland Reagents (800) 247-8766, or Ransom Hill (800) 597-8509. The Competimer™ may have the same or essentially the same sequence as a primer employed in the synthesis reaction, but for a 3′ terminal hydroxyl group which has been modified in a manner that prevents the extension of the primer by a polymerase. See U.S. Pat. No. 6,057,134 (incorporated by reference in whole and in part) for further characteristics of Competimers™ and their uses, including at col. 3, line 50 to col. 6, line 10, Example 8, and/or in the claims.

2. Binding Efficiency

In a further embodiment, the distinguishing tags (including distinguishable probes and distinguishable primers) have different relative binding efficiencies to the target nucleic acid. Factors that affect hybridization, outlined above, can affect the kinetics and/or equilibrium of the nucleic acid binding reaction, and thus the binding efficiency. For example, hybridization and the strength of hybridization is influenced by such factors as the degree of complementarity between the nucleic acids, the length of the hybridizing portion of nucleic acid, the stringency of the hybridization conditions involved, the melting temperature (Tm) of the formed hybrid, and the G:C ratio within the nucleic acids. In some embodiments, a distinguishable primer has a lower melting temperature, reflecting a shift in the equilibrium of the binding. Altering the binding efficiency and/or Tm of binding can be accomplished by changing (e.g., decreasing) the length of the target binding sequence or hybridizing portion of a distinguishing tag, for example. In one embodiment, primers with overlapping sequences, but which vary in length (e.g. by extending the length of the primer binding site at the 3′ and/or 5′ ends) will have different relative binding efficiencies to the target nucleic acid.

In general, any base or sugar modification that creates unfavorable interactions and/or disrupts the natural geometry of RNA:RNA, RNA:DNA, or DNA:DNA interactions may result in reduced binding efficiency. Modified bases or nucleotides within a nucleic acid can increase or decrease the binding efficiency of that nucleic acid for a complementary nucleic acid. Some modifications may alter the position of hybridization equilibrium; others may decelerate the rate of hybridization without significantly affecting the equilibrium point, for example. In the latter case, such destabilization of nucleic acid interactions may still be exploited in the invention inasmuch as the hybridization time can be determined to exacerbate differences between the on-rates of various hybridizing species. Without being limited to any chemical entity or class of nucleic acid modifications, destabilization modifications may include 2-thiolated and 4-thiolated uridine, thymidine, and cytidine bases, 6-methyl adenosine, 4-ethylcytidine, and various bases such as inosine, nitropyrrole, and nitroindole. Modified nucleotides and modified bases can be included to increase binding affinity to the target or to reduce binding affinity to the target, for example.

DNA and RNA are polynucleotides that include deoxyriboses or riboses, respectively, coupled by phosphodiester bonds. Each deoxyribose or ribose includes a base coupled to a sugar. The bases incorporated in naturally-occurring DNA and RNA are adenosine (A), guanosine (G), thymidine (T), cytidine (C), and uridine (U). These five bases are “natural bases.” According to the rules of base pairing elaborated by Watson and Crick, the natural bases can hybridize to form purine-pyrimidine base pairs, where G pairs with C and A pairs with T or U. These pairing rules facilitate specific hybridization of an oligonucleotide with a complementary oligonucleotide.

The formation of these base pairs by the natural bases is facilitated by the generation of two or three hydrogen bonds between the two bases of each base pair. Each of the bases includes two or three hydrogen bond donor(s) and hydrogen bond acceptor(s). The hydrogen bonds of the base pair are each formed by the interaction of at least one hydrogen bond donor on one base with a hydrogen bond acceptor on the other base. Hydrogen bond donors include, for example, heteroatoms (e.g., oxygen or nitrogen) that have at least one attached hydrogen. Hydrogen bond acceptors include, for example, heteroatoms (e.g., oxygen or nitrogen) that have a lone pair of electrons.

The natural bases A, G, C, T, and U, can be derivatized by substitution at non-hydrogen bonding sites to form modified natural bases. For example, a natural base can be derivatized for attachment to a support by coupling a reactive functional group (e.g., thiol, hydrazine, alcohol, or amine) to a non-hydrogen bonding atom of the base. Other possible substituents include biotin, digoxigenin, fluorescent groups, and alkyl groups (e.g., methyl or ethyl).

Non-standard bases, which form hydrogen-bonding base pairs, can also be constructed as described, for example, in U.S. Pat. Nos. 5,432,272, 5,965,364, 6,001,983, and 6,037,120 and U.S. patent application Ser. No. 08/775,401, all of which are incorporated herein by reference. By “non-standard base” it is meant a base other than A, G, C, T, or U that is susceptible of incorporation into an oligonucleotide and which is capable of base-pairing by hydrogen bonding, or by hydrophobic, entropic, or van der Waals interactions to form base pairs with a complementary base. Specific examples of these bases include the following bases, iso-guanine (iso-G), iso-cytosine (iso-C), xanthine (X), kappa (K), nucleobase H, nucleobase J, nucleobase M, and nucleobase N (see U.S. Pat. No. 6,001,983), in base pair combinations (iso-C/iso-G, K/X, H/J, and M/N). It will be recognized that other non-standard bases utilizing hydrogen bonding can be prepared, as well as modifications of the above-identified non-standard bases by incorporation of functional groups at the non-hydrogen bonding atoms of the bases.

The hydrogen bonding of these non-standard base pairs is similar to those of the natural bases where two or three hydrogen bonds are formed between hydrogen bond acceptors and hydrogen bond donors of the pairing non-standard bases, for example. One of the differences between the natural bases and these non-standard bases is the number and position of hydrogen bond acceptors and hydrogen bond donors. For example, cytosine can be considered a donor/acceptor/acceptor base with guanine being the complementary acceptor/donor/donor base. Iso-C is an acceptor/acceptor/donor base and iso-G is the complementary donor/donor/acceptor base, as illustrated in U.S. Pat. No. 6,037,120, incorporated herein by reference.

Other non-standard bases for use in oligonucleotides include, for example, naphthalene, phenanthrene, and pyrene derivatives as discussed, for example, in Ren et al., J. Am. Chem. Soc. 118:1671 (1996) and McMinn et al., J. Am. Chem. Soc. 121:11585 (1999), both of which are incorporated herein by reference. These bases do not utilize hydrogen bonding for stabilization, but instead rely on hydrophobic, entropic, or van der Waals interactions to form base pairs.

B. Differentially Amplifying Distinguishable Amplicons

In some embodiments of the invention, the dynamic range of detection or of quantification (e.g. linearity) can be expanded by amplifying different distinguishable amplicons at different amplification efficiencies, which results in different amounts of amplified nucleic acids from the distinguishable amplicons. The different amounts of amplified nucleic acids are then detected and quantitated. By attenuating the amplification efficiencies in a step-wise manner over a broad range of efficiencies, at least one of the amplified nucleic acid amounts will be detectable within the dynamic range of the detection method, even when the approximate amount of the target nucleic acid in a sample is not previously known. Because each of the distinguishable amplicons is amplified by a unique amplification primer set independent of other primers used for other distinguishable amplicons, multiple distinguishable amplicons of the same target can be amplified in a single reaction.

In certain embodiments the primer for synthesis (e.g. amplification) can be a universal primer segment such as a segment that corresponds to a primer that can be used to prime 2, 3, 4, 5, 6, or more different amplicons from the same or different DNA or RNA targets or subsequences of targets. In some embodiments the primer is specific for a target RNA or DNA. The primer can be at least or about 5, 6, 7, 8, 9, 10, 11, 12, 13, 14, 15, 16, 17, 18, 19, 20, 21, 22, 23, 24, 25, 26, 27, 28, 29, 30, 40, 50, 100, or more nucleotides in length, including all values and ranges there between.

A zip code portion will typically be distinct from a primer segment and/or the target specific segment of an RT primer, for example. A zip code acts as a label, probe or probe segment, in many embodiments. A distinguishable probe may comprise a zip code. The zip code can be at least or about 5, 6, 7, 8, 9, 10, 11, 12, 13, 14, 15, 16, 17, 18, 19, 20, 21, 22, 23, 24, 25, 26, 27, 28, 29, 30, 40, 50, 100, or more nucleotides in length, including all values and ranges there between. In certain aspects the zip code will be adjacent to the primer, the target specific segment, or both the primer segment and the target specific segment.

1. Primer Dilution and Competitive Binding

In one embodiment, the amplification efficiency of the distinguishable amplicons is attenuated by adding different concentrations of amplification primers to the amplification reaction. Thus one distinguishable amplicon can be amplified using a set of primers at one concentration, while other distinguishable amplicons can be amplified using primers of a different concentration, resulting in different concentrations of amplified nucleic acids. Primers may be diluted by at least or about 5-fold, 10-fold, 25-fold, 50-fold, 75-fold, 100-fold, 200-fold, 300-fold, 400-fold, 500-fold, 750-fold, 1000-fold, 10000-fold, 50000-fold, 100000-fold, (including all values and ranges there between), for example to attenuate the amplification efficiency of a reaction.

Reactions are carried out with suitable reaction components, including buffers, salts, enzymes, nucleotides, and optionally one or more passitivation agents, such as a non-specific nucleic acid. Surface passitivation may occur with Poly(A) RNA, salmon sperm DNA, yeast total RNA, yeast tRNA, E. coli total RNA, a polynucleotide analog, a polymer, or other suitable passivitation reagent or method.

In one embodiment, the amplification efficiency of the distinguishable amplicons is attenuated by adding one or more competimers at the same or different concentrations to the amplification reaction. A competimer is a nucleic acid primer that hybridizes to the same sequence or an overlapping sequence as a primer but is blocked at the 3′ terminus and thus cannot be extended in primer-dependent enzymatic reactions. Different concentrations of competimers to different distinguishable amplicons can be used to produce different amounts of amplified products.

2. Binding Efficiency

In another embodiment, the amplification efficiency is attenuated by altering the binding efficiency of the amplification primers to the distinguishable amplicons, so that different relative binding efficiencies affect differential amplification. Methods of altering binding efficiency are discussed above. In one specific embodiment, the length of one or more amplification primers is chosen such that at least two primer melting temperatures are different. In another, the primer includes modified nucleotides, and in another embodiment, the primer comprises a mismatch in the base-pairing with the amplicon or target sequence.

In a further embodiment, the amplification efficiency is attenuated by competitive hybridization, including by adding competing oligonucleotides to the amplification mixture. As discussed above, these oligonucleotides comprise a sequence similar or identical to a portion of the sequence of the distinguishable amplicons or distinguishing tags and therefore compete with the distinguishable amplicons for binding to an amplification primer. In an embodiment, the competing sequence comprises a complementary sequence to one of the amplicon-specific primers.

In yet another embodiment, the amplification efficiency can be attenuated by selecting for amplified nucleic acids having different concentrations of single stranded nucleic acids. For example, asymmetric PCR can be used to disproportionally increase the total amount of single stranded product that is amplified (Gyllensten et al., Proc. Natl. Acad. Sci. USA 85:7652-7656 (1988). By comparison, symmetrical PCR generates a double stranded product. Since nucleic detection is often achieved by hybridization of complementary probe sequences, a double stranded product typically requires dissociation of the complementary strand to enable hybridization of the probe. Thus, amplicons can be distinguishable at the hybridization step by signal attenuation that increases with an increasing fraction of double stranded character present in the amplicons that are differently detected.

3. Exemplary Embodiments

A first exemplary embodiment of the multiplex technology uses two or more probes that are homologous to the same target sequence. The probes contain a non-homologous sequence adjacent to the target sequence homologous region. This non-homologous sequence contains a “zip code” sequence that is different for each probe and uniquely identifies the probe. Flanking both sequences are additional sequences which can be used to amplify the probes (primer binding sites). The probes (immobilized on filters or beads) are hybridized to the target. After hybridization, the unhybridized nucleic acid is washed away so that only probe bound to target remains.

If in vitro transcription is used to amplify the probe, then a promoter sequence will be used on only one side of the probe-zip code sequence. For example, the probe-zip code sequence could be flanked by a phage polymerase promoter such as T3, T7, or Sp6. Clearly other amplification technologies where the amplification is mediated by a primer binding to a specific sequence could be used instead.

If PCR is used to amplify the probes and each probe has differing PCR primers, then the amplification of each probe will be largely independent of the others. If three differing probes (that differ in the zip code and primer binding sites) are used, there will be three largely independent determinations of the amount of the target. If these experimental values are averaged together, this will have the effect of increasing the accuracy of the determination of the amount of target.

If, however, the amplification efficiency of the three amplicons is different, then this will have the effect of increasing the dynamic range of the assay. For example, each of the three probes is present in equal amounts, and there are equal amounts of PCR primers for each probe (n.b. the PCR primers for each probe differ in sequence and will only amplify one of the probes). For two of the probes, in addition to the PCR primers, there are PCR primers that are blocked at the 3′ end. These are referred to as “competimers.” They are non-functional in a PCR reaction and compete with the conventional primers to reduce the efficiency of the amplification reaction. Competimers or a titration of competimers may be used in multiplex PCR reactions to reduce the amplification efficiency of a very abundant target to allow quantification, for example in the same reaction as quantifying a less abundant target.

In the present exemplary embodiment, sufficient competimer #1 is added to the reaction to reduce the amplification efficiency 100 fold relative to the reaction with no competimer. For the second amplicon, sufficient competimer #2 is added to the reaction to reduce the amplification efficiency 10 fold relative to the reaction with no competimer. For the third amplicon, no competimer is added. If the PCR reaction is stopped prior to the plateau phase, there will be three differing amounts of PCR product that differ in amount by a factor of 10. Many methods of detecting PCR reaction products have a dynamic range of less than a 1000 fold. The use of three different amplicons whose amplification efficiencies vary by factors of 10 will increase the effective dynamic range of the reaction by a factor of 100.

The zip code sequences can be used to detect each of the three different amplicons. This could be done by using zip codes which differ in length so that the PCR products can be detected on a gel. In some methods, the zip codes will differ in sequence and can be used to hybridize the amplification products to an array. In certain methods the array will be a bead array such as is utilized in the Luminex instrument. This instrument has the capability of distinguishing 100 different beads allowing the independent determination of 100 different analytes in the same reaction well. In the present example three different Luminex beads would have three different sequences attached to them that were complementary to the reaction products from the PCR amplification reaction described above. The amount of each reaction product would differ by about a factor of 10. Thus, the amount of PCR product from reaction number three from above would be present at the highest level. It might be an amount which saturates the detection limit of the beads and the instrument. The amount of PCR product from reaction number two would be present at one tenth the amount and might not saturate the detection limit. If the amount of PCR product from reaction number two still was present at too high a level, then the PCR product from the first amplicon would be present at the lowest level. Any one of the three PCR products could be used to quantify the amount of target. This technology is particularly useful where the target amount in a sample varies significantly over a wide range and/or in situations in which it is not convenient to separately titrate the assay or sample.

In an additional amplification method using a straight PCR reaction, an inner and an outer set of primers amplify a DNA target. The inner primer set is used for one or a few cycles of amplification. Later cycles use an outer set of primers that differentially amplify the differing products of the first inner set of primers whose purpose is to produce products which differ in sequence at one end of the product. The first inner PCR primer on one side of the sequence is a conventional PCR primer, for example. The inner PCR primer on the other side consists of three different PCR primers that compete with each other for a common binding site on the target. In the 5′ direction, these primers have differing zip codes. In a method, the primers have differing sequences for additional outer PCR primers to bind further in the 5′ direction. Thus, the reaction has two steps. In the first step three amplification products are formed from the target. The products differ at one end by having differing zip codes and differing 5′ end sequences which can be used as primer binding sites for a secondary PCR reaction. In practice there might be only a limited amount of the inner primers that would be largely consumed in the first few PCR cycles. Alternatively, the inner PCR primers might be designed to have a lower melting temperature than the outer primers. The first few cycles would have an annealing temperature that was low enough that the inner primers would be capable of forming amplification products. The annealing temperature would then be raised to a level where only the outer primers would be capable of forming amplification products. Another alternative would be to add outer primers after the first few cycles of the PCR.

C. Differentially Detecting Distinguishable Amplicons

In some embodiments of the invention, the dynamic range of the nucleic acid quantification method can be expanded by differentially detecting amplified nucleic acids from distinguishable amplicons. By attenuating the detection of the amplified nucleic acids in a step-wise manner over a broad range of concentrations, at least one of the amplified nucleic acid amounts will be detectable within the dynamic range of the detection method. Exemplary detection methods, techniques, and labels are described above, and include branched DNA detection and fluorescent in-situ hybridization, for example.

In one embodiment, the amplified nucleic acids are differentially detected using probes with different binding efficiencies to the amplified nucleic acids. It is to be understood that because the distinguishable amplicons comprise distinguishing tags that allow the distinguishable amplicons to be differentiated from one another, nucleic acids amplified from the distinguishable amplicons will also comprise the distinguishing tags, which can be used to individually detect the distinguishable amplicons. Thus, a probe to one distinguishable amplicon can have a different binding efficiency than a probe to another distinguishable amplicon. Differential binding is described above.

In another embodiment, the amplified nucleic acids are differentially detected using competitive hybridization to oligonucleotides. In a first competitive hybridization scheme, oligonucleotides that are similar or identical to the amplified nucleic acid are added to the detection assay. In this case, the oligonucleotides compete with the amplified nucleic acid for binding with the detection probe. In a second competitive hybridization scheme, oligonucleotides that are similar or identical to the detection probe are added to the detection assay. In this case, the oligonucleotide competes with the probe for binding to the amplified nucleic acids.

III. Kits

The invention includes kits of reagents and macromolecules for carrying out assays according to this invention. In an embodiment, the invention provides a kit for detecting a target nucleic acid sequence in a sample comprising multiple distinguishing tags, wherein at least two of the distinguishing tags comprise: (a) a target binding sequence that is complementary to a target nucleic acid sequence; and b) a zip code that uniquely identifies the distinguishing tag. In certain aspects, the distinguishing tags further comprise a primer binding site and/or a distinguishable primer binding site. The kits optionally further comprise standard reagents, such as target nucleic acid molecules, distinguishing tags, primers, or probes. The kits further optionally comprise an enzyme for carrying out the assays described herein, including but not limited to a polymerase such as a reverse transcriptase or a DNA polymerase, or a ligase. The kits optionally include nucleic acid sequences or subsequences that are identical or complementary to the assay target(s) that are provided in known quantities. Such molecules may serve as absolute standards for creating standard curves to quantify the unknown levels of target in the sample of interest. In various aspects, the kits may comprise multiple amplification primer sets, wherein at least one of the primers in each of the primer sets comprises a sequence that is complementary to a portion of at least one distinguishing tag. In other aspects, the kits further comprise at least two probes complementary to a portion of at least two distinguishing tags.

Any of the compositions or reagents described herein may be comprised in a kit. In a non-limiting example, reagents for reverse transcribing a RNA target, for example to obtain multiple distinguishable amplicons, using a RT primer comprising in a 5′ to 3′ direction a primer segment, a probe segment, and a target specific annealing segment are included in a kit. The kit may also include multiple RT primers to multiple sites on one or more RNA. The kit may also comprise reagents for reverse transcribing RNA to a DNA template and/or reagents, including primers, for amplification of the target DNA. Such a kit may include one or more buffers, such as a reaction, amplification, and/or a transcription buffer, compounds for preparing a RNA sample, for preparing a DNA sample, and components for isolating and/or detecting an amplification product, such as probe or label, for example.

In some embodiments, kits of the invention include one or more of the following in a suitable container (consistent with methods, reagents, and compositions discussed above): components for sample purification, including a lysis buffer with a chaotropic agent; a glass fiber filter or column; elution buffer; wash buffer; alcohol solution; and nuclease inhibitor are optionally included.

The components of the kits may be packaged either in aqueous media or in lyophilized form. The containers will generally include at least one vial, test tube, flask, bottle, syringe, or other container means, into which a component may be placed, and preferably, suitably aliquoted. Where there is more than one component in the kit (they may be packaged together), the kit also will generally contain a second, third, or other additional container into which the additional components may be separately placed. However, various combinations of components may be comprised in a vial. The kits of the present invention will also typically include a means for containing the RNA, and any other reagent containers in close confinement for commercial sale. Such containers may include injection or blow-molded plastic containers into which the desired vials are retained. When the components of the kit are provided in one and/or more liquid solutions, the liquid solution is an aqueous solution, with a sterile aqueous solution being particularly preferred.

Alternatively, the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe, and/or other container means, into which the nucleic acid formulations are placed, preferably, suitably allocated. The kits may also comprise a second container means for containing a sterile, pharmaceutically acceptable buffer and/or other diluent.

Such kits may also include components that preserve or maintain DNA or RNA or that protect against its degradation. Such components may be nuclease or RNase-free or protect against RNases. Such kits generally will comprise, in suitable means, distinct containers for each individual reagent or solution.

### IV. Libraries and Arrays

The present methods and kits may be employed for high volume screening. A library of either DNA or RNA can be created and used in high throughput assays, including microarrays, according to the methods of the invention. Specifically contemplated by the present inventors are chip-based nucleic acid technologies such as those described by Hacia et al. (1996) and Shoemaker et al. (1996). Briefly, these techniques involve quantitative methods for analyzing large numbers of genes rapidly and accurately. By using fixed probe arrays, one can employ chip technology to segregate target molecules as high density arrays and screen these molecules on the basis of hybridization (see also, Pease et al. (1994); and Fodor et al. (1991)).

The term “array” as used herein refers to a systematic arrangement of nucleic acid. For example, a nucleic acid population that is representative of a desired source (e.g., human adult tissue) is divided up into the minimum number of pools in which a desired screening procedure can be utilized to detect or deplete a target gene and which can be distributed into a single multi-well plate. Arrays may be of an aqueous suspension of a nucleic acid population obtainable from a desired mRNA source, comprising: a multi-well plate containing a plurality of individual wells, each individual well containing an aqueous suspension of a different content of a nucleic acid population. Examples of arrays, their uses, and implementation of them can be found in U.S. Pat. Nos. 6,329,209; 6,329,140; 6,324,479; 6,322,971; 6,316,193; 6,309,823; 5,412,087; 5,445,934; and 5,744,305, which are herein incorporated by reference.

A. Bead Arrays

In one embodiment, the multiple distinguishable amplicons can be detected using a bead array platform. In bead array systems, probes to specific target sequences (or other targets of interest) are attached to beads suspended in solution. The beads contain a “barcode” that allows each bead to be differentially detected. In one example, the beads are filled with fluorescent dyes that allow each bead to have a unique spectral identity. Therefore, specific probes can be correlated with beads having a specific spectral signature. The beads are contacted to a solution or sample containing target sequences or analytes suspended in the sample, and they are allowed to hybridize. Non-specifically bound materials are washed away. In some types of bead array systems, the target sequences or analytes can be labeled prior to binding with the beads. Therefore, once a target sequence or analyte has bound to a bead, the bound bead can be detected by screening for the label. In other bead array systems, when a target sequence or analyte binds to a probe on a bead, this causes a reporter channel to fluoresce, which allows detection of a bound bead. An example of a bead array system is the Luminex xMAP platform that uses fluorescent dye-filled beads.

B. Planar Arrays

In another embodiment, the multiple distinguishable amplicons can be detected using planar arrays. In planar array systems, probes to specific targets are fixed to a solid support, and samples containing target sequences or other molecules are applied to the arrays. Methods of making planar arrays, including methods of affixing probes to solid supports, are known to persons skilled in the art. As with bead array systems, nucleic acids can be labeled before binding to probes. Alternatively, the bound complex may be detected. Thus, bound probes are located by detecting the presence of labels bound to the array.

Microarrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” is a nucleic acid or nucleic acid analogue to which a particular cognate cDNA can specifically hybridize. The nucleic acid or analogue of the binding site can be, e.g., a synthetic oligomer, a full-length cDNA, a less-than-full-length cDNA, an RNA, or a nucleic acid fragment.

The nucleic acid or analogue is attached to a solid support. Particles can be fabricated from virtually any insoluble or solid material. For example, the particles can be fabricated from silica gel, glass, nylon, resins, Sephadex™, Sepharose™, cellulose, magnetic material, a metal (e.g., steel, gold, silver, aluminum, copper, or an alloy) or metal-coated material, a plastic material (e.g., polyethylene, polypropylene, polyamide, polyester, polyvinylidenefluoride (PVDF) and the like), and combinations thereof. Examples of suitable micro-beads are described, for example, in U.S. Pat. Nos. 5,736,330, 6,046,807, and 6,057,107, all of which are incorporated herein by reference. Examples of suitable particles are available, for example, from Luminex Corp., Austin, Tex.

Nucleic acids may be directy attached to a surface or indirectly attached, and they may optionally be attached via a linker sequence or molecule. A preferred method for attaching the nucleic acids to a surface is by printing on glass plates. Other methods for making microarrays, e.g., by masking, may also be used. In principal, any type of array, for example, dot blots on a nylon hybridization membrane (see Sambrook et al., 1989, which is incorporated in its entirety for all purposes), could be used, although, as will be recognized by those of skill in the art, very small arrays will be preferred because hybridization volumes will be smaller.

### V. Diagnostic Methods

Methods of the invention are applicable to any number of applications where it is desirable to determine an amount of a target nucleic acid sequence. The methods can be used in analytical or diagnostic applications.

In some embodiments, the methods of the invention are applicable to methods for diagnosing and/or assessing a disease, condition, or potential condition in a patient comprising determining an amount of a target nucleic acid sequence in a sample from the patient. In some embodiments, the amount of a target nucleic acid in the sample is indicative of the presence or absence of a disease, the disease progression, prognosis, or risk thereof. In other embodiments, the detection of a target nucleic acid in the sample is the diagnostic or prognostic indicator, for example.

A “patient sample” is any biological specimen derived from a patient. The term includes, but is not limited to, biological fluids such as blood, serum, plasma, urine, cerebrospinal fluid, tears, saliva, lymph, dialysis fluid, lavage fluid, semen, and other liquid samples, as well as cell and tissues of biological origin. The term also includes cells or cells derived therefrom and the progeny thereof, including cells in culture, cell supernatants, and cell lysates. It further includes organ or tissue culture-derived fluids, tissue biopsy samples, tumor biopsy samples, tissue prints (cells affixed to a solid substrate such as nitrocellulose and optionally obtained by pulling off a thin layer of cells from a tissue sample), stool samples, and fluids extracted from physiological tissues, as well as cells dissociated from solid tissues, tissue sections, and cell lysates. This definition encompasses samples that have been manipulated in any way after their procurement, such as by treatment with reagents, solubilization, or enrichment for certain components, such as polynucleotides or polypeptides. Also included in the term are derivatives and fractions of patient samples. A sample may be taken from a patient having, suspected of having, or recovering from a disease or pathological condition. The sample can be fresh, frozen, fixed (e.g., formalin fixed), or embedded (e.g., paraffin embedded).

The present invention is of particular interest in the diagnostic screening of samples for many diseases or conditions. In certain embodiments, diagnostic methods involve identifying one or more nucleic acids, such as miRNAs or mRNA transcripts, in a sample that are indicative of a disease or condition. In certain embodiments, diagnosing a disease or condition involves detecting and/or quantifying an expressed nucleic acid, such as miRNA or mRNA. Nucleic acids clearly linked to a disease phenotype are referred to as “biomarkers.”

The methods may be used to guide therapeutic decision-making and other aspects of disease management. The methods may also be used to detect disease-specific nucleic acids or biomarkers (e.g., translocations, mutations, fusion transcripts, increases or decreases in expression, etc.). For example. in diseases such as leukemia, (including acute lymphocytic leukemia (ALL), acute myeloid (myelogenous) leukemia (AML), chronic lymphocytic leukemia (CLL), and chronic myelogenous leukemia (CML), the knowledge of precise molecular alterations, such as specific translocations, can have prognostic value. In some embodiments, the invention is useful in the detection of a of biomarker in a patient where there is a correlation between the amount of biomarkers and disease presence or clinical outcome. In one embodiment, the method of the invention is useful for the detection of BCR-ABL (breakpoint cluster region-abelson) transcripts in chronic myeloid leukemia patients, where there is a positive a correlation between the level of this fusion transcript and outcome.

In certain embodiments, the methods detect the presence, absence, up-regulation, or down-regulation of certain target nucleic acid sequences in treated cells, cell lines, tissues, or organisms. Embodiments of the invention include methods for diagnosing and/or assessing a condition or potential condition in a patient comprising determining the amount of a target nucleic acid sequence in a sample from a patient. The difference in the nucleic acid in the sample from a patient and the nucleic acid in a reference sample (e.g. a normal or non-pathologic sample), is indicative of a pathology, disease, or cancerous condition, or risk thereof, for example. The invention may also be used for the detection of nucleic acids that are indicative of infectious disease, such viral, fungal, or bacterial infections.

A “disease” is a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases. Diseases also include neural, immune system, muscular, reproductive, gastrointestinal, pulmonary, cardiovascular, renal, proliferative, and/or cancerous diseases, disorders, and conditions.

Particularly the methods can be used to evaluate samples with respect to diseases or conditions that include, but are not limited to: Alzheimer's disease, macular degeneration, chronic pancreatitis; pancreatic cancer; AIDS, autoimmune diseases (rheumatoid arthritis, multiple sclerosis, diabetes-insulin-dependent and non-independent, systemic lupus erythematosus and Graves disease); proliferative disorder (e.g., cancer, malignant cancer, benign, metastatic, precancer); cardiovascular diseases (heart disease or coronary artery disease, stroke-ischemic and hemorrhagic, and rheumatic heart disease); diseases of the nervous system; inflammation (allergy, asthma); prion diseases (e.g., CJD, kuru, GSS, FFI); and infection by pathogenic microorganisms (e.g. bacteria, viruses, protists, and fungi; and exemplified by diptheria, chickenpox, common cold, diarrheal diseases, flu, genital herpes, malaria, meningitis, pneumonia, dinusitis, skin diseases, strep throat, tuberculosis, urinary tract infections, vaginal infections, viral hepatitis).

Exemplary cancers include hematologic malignancies, leukemia (including acute leukemias (for example, acute lymphocytic leukemia, acute myelocytic leukemia, including myeloblastic, promyelocytic, myelomonocytic, monocytic, and erythroleukemia)) and chronic leukemias (for example, chronic myelocytic (granulocytic) leukemia and chronic lymphocytic leukemia), myelodysplastic syndrome polycythemia vera, lymphomas (for example, Hodgkin's disease and non-Hodgkin's disease), multiple myeloma, Waldenstrom's macroglobulinemia, heavy chain diseases, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, glandular carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, thyroid carcinoma or sarcoma, lung carcinoma, small cell lung carcinoma, bladder cancer, skin cancer (including, e.g., epithelial carcinoma, sarcoma, and melanoma), glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma; and/or cancer metastases, including metastases in bone, liver, and lung.

In addition, the cancer may specifically be of a following type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinorna, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; rnucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Moreover, RNA and DNA can be evaluated in pre-cancers, such as metaplasia, dysplasia, and hyperplasia.

It is specifically contemplated that the invention can be used to evaluate differences between stages of disease, such as between hyperplasia, neoplasia, pre-cancer, and cancer, or between a primary tumor and a metastasized tumor.

### EXAMPLES

The following examples illustrate various embodiments of the invention and are not intended to limit the scope of the invention.

### Example 1

### Multiplex Quantification of a Target Nucleic Acid Sequence

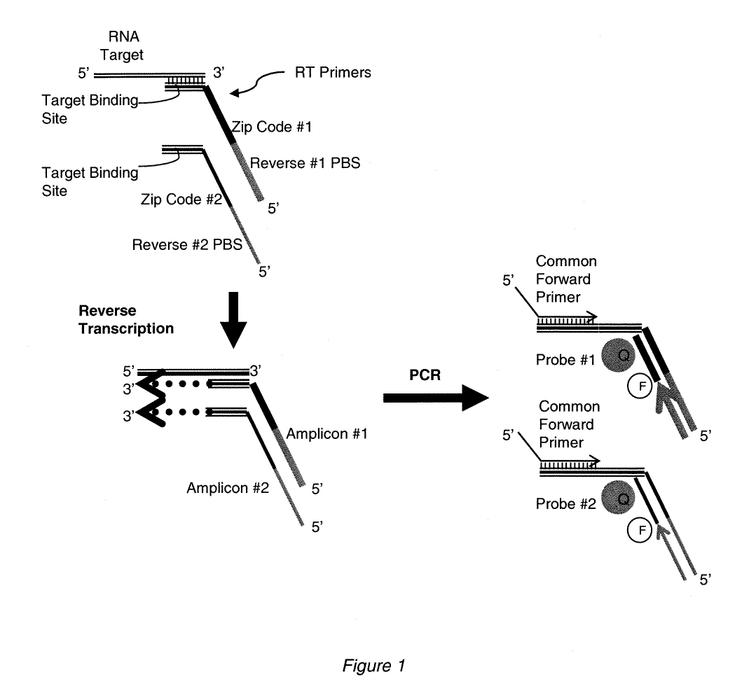

To test the concept of independent and attenuated quantification of multiple amplicons produced from the same target sequence, the following model system was designed (see schematic in FIG. 1). A synthetic RNA corresponding to let 7a miRNA was synthesized (Integrated DNA Technologies). Reverse transcription (RT) primers were designed comprising three discrete segments: i) a sequence complementary to the 3′ region of the miRNA; ii) a segment encoding a sequence non-complementary to the target that was unique to each RT primer (the “zip code”); and iii) a segment encoding a sequence non-complementary to the target but complementary to a reverse PCR primer that was unique to each RT primer. A forward primer complementary to the 5′ region of the target miRNA was also designed. Amplification thus produces two amplicons from the single target that can be distinguished by the use of unique reverse primer binding sites and/or zip code sequence tags. For experiments evaluating methods to attenuate amplification efficiency, a dual-labeled fluorescence probe (TaqMan™ probe) bearing the respective zip code sequences was used to permit amplicon quantification. The quantification may be accomplished via real-time RT-PCR using methods known to those skilled in the art.

### Example 2

### Dynamic Range Expansion Using Different Primer Concentrations in RT Reaction

One strategy for reducing the signal from abundant targets after amplification is to dilute the RT primer that seeds cDNA synthesis. As a result, less cDNA is made, and thus less of the starting amplicon is passed into PCR. The net effect of this change at the level of PCR is an increase in the offset of the PCR standard curve, without a change in the slope of the curve. To demonstrate the utility of this approach, multiplex RT reactions were prepared using two different RT primers directed against a single let 7a target as described in Example 1. The RT primer sequences contained an identical target annealing region, a unique probe sequence upstream of the annealing region, and a unique reverse PCR primer sequence upstream of the probe sequence. RT primer sequences are shown in Table 1 below. In the table, let 7a target binding sequences are capitalized without underlining. Unique zip code sequences are shown in lower case. Unique reverse PCR primer sequences are capitalized and underlined. The let 7a target sequence is: ugagguaguagguuguauaguu (SEQ ID NO:1).

TABLE 1
RT PRIMER SEQUENCES FOR EXAMPLE 2

| | | |
|---|---|---|
| RT-1 | 5′-GGTCCGACTACCCCAACAAtaccttgaaccctacagcag | SEQ ID NO: 2 |
| | agtACTATACAAC-3′ | |
| RT-2 | 5′-CAAAGGCTGCCAACATAAAATGcaagcgtaaatgcagc | SEQ ID NO: 3 |
| | gtccaACTATACAAC-3′ | |

Reverse transcription components were assembled on ice prior to the addition of the RNA template and are listed in Table 2 below. The RT-1 RT primer includes zip code sequence 1, whereas the RT-2 RT primer includes zip code sequence 2. These two zip code sequences were embedded in two corresponding TaqMan™ probes labeled with either FAM (carboxyfluoroscein) or VIC (a fluorophore produced by Applied Biosystems Inc.) to enable simultaneous detection of both amplicons during the amplification reaction. The RT-2 primer concentration was tested over a range of decreasing concentrations including 50, 5, 0.5, 0.1, 0.05, and 0.01 nM whereas the RT-1 primer concentration remained at 50 nM.

TABLE 2
REVERSE TRANSCRIPTION REACTION COMPONENTS FOR
EXAMPLE 2

| | μl per | Final |
|---|---|---|
| Component | 10 μl rxn | Concentration |
| Nuclease-free water | 3.80 | |
| 10X RT Buffer (RETROscript ™, | 1.00 | 1X |
| Ambion) | | |
| dNTP mix (2.5 mM each) (Ambion) | 1.00 | 0.25 mM each |
| RT Primer-1 (500 nM) | 1.00 | 50 nM |
| RT Primer-2 (500 nM-0.1 nM) | 1.00 | 50 nM to 0.01 nM |
| RNase Inhibitor (40 U/μl) (Ambion) | 0.10 | 0.4 U/μl |
| Moloney Murine Leukemia Virus | 0.10 | 1 U/μl |
| Reverse Transcriptase | | |
| (MMLV-RT) (100 U/μl) (Ambion) | | |
| Synthetic let 7a RNA | 2.00 | |

Following assembly of the reaction components on ice, the RNA template (1e5-1e10 copies (1×105 to 1×1010) of a synthetic mature let 7a RNA) was added in a background of 10 ng/μl of Poly(A) RNA. Poly(A) RNA is included to passivate the surface of the reaction tube and ensure the solution availability of the synthetic RNA template for enzymatic manipulation during the multiplex RT-PCR reaction. The source of polyadenylic acid (Poly(A)) is Sigma-Aldrich and is prepared from ADP with polynucleotide phosphorylase to create a heterogeneous polymer of adenylic acid. The reverse transcription reaction was incubated in a 384-well GeneAmp™ PCR System 9700 (Applied Biosystems) at 16° C. for 15 min, then at 42° C. for 15 min, then at 85° C. for 10 min, then transferred to ice.

The reaction components for PCR are shown in Table 3 and were assembled on ice prior to the addition of the cDNA from the reverse transcription reaction. PCR primer sequences are shown in Table 4. TaqMan™ probes were dual-labeled with 5′-VIC and 3′-MGB-NFQ (minor groove binder non-fluoroscent quencher) or 5′-6-FAM and 3′-TAMRA (tetramethyl-6-carboxyrhodamine).

TABLE 3
REAL-TIME PCR COMPONENTS FOR EXAMPLE 2

| | μl per | Final |
|---|---|---|
| Component | 15 μl rxn | Concentration |
| Nuclease-free water | 5.65 | |
| MgCl₂ (50 mM) | 1.50 | 5 mM |
| 10X Platinum PCR Buffer, Minus Mg | 1.50 | 1X |
| (Invitrogen) | | |
| dNTP mix (2.5 mM each) | 1.50 | 0.25 nM each |
| let 7a Forward 13 Primer (10 μM) | 0.75 | 500 nM |
| Rev-1 (specific to Zip code 1) (10 μM) | 0.75 | 500 nM |
| Rev-2 (specific to Zip code 2) (10 μM) | 0.75 | 500 nM |
| RT-1 VIC MGB TaqMan ™ Probe (2 μM) | 0.60 | 80 nM |
| RT-2 FAM TAMRA TaqMan ™ Probe | 0.60 | 80 nM |
| (2 μM) | | |
| 50X ROX Internal marker | 0.30 | 1X |
| Platinum Taq (5 U/μl) | 0.10 | 0.033 U/μl |
| cDNA from RT reaction | 1.00 | |

TABLE 4
PCR PRIMER SEQUENCES FOR EXAMPLE 2

| | | |
|---|---|---|
| let 7a | 5′-CGGCGCGTGAGGTAGTAGGT-3′ | SEQ ID NO: 4 |
| Forward | | |
| Primer | | |
| Rev-1 | 5′-GGTCCGACTACCCCAACAA-3′ | SEQ ID NO: 5 |
| Reverse | | |
| Primer | | |
| Rev-2 | 5′-CAAAGGCTGCCAACATAAAATG-3′ | SEQ ID NO: 6 |
| Reverse | | |
| Primer | | |
| RT-1 VIC | 5′-VIC-CTTGAACCCTACAGCAGAGT- | SEQ ID NO: 7 |
| MGB | MGB-NFQ-3′ | |
| Taq- | | |
| Man ™ | | |
| Probe | | |
| RT-2 FAM | 5′-FAM-CAAGCGTAAATGCAGCGTCCA- | SEQ ID NO: 8 |
| TAMRA | TAMRA-3′ | |
| Taq- | | |
| Man ™ | | |
| Probe | | |

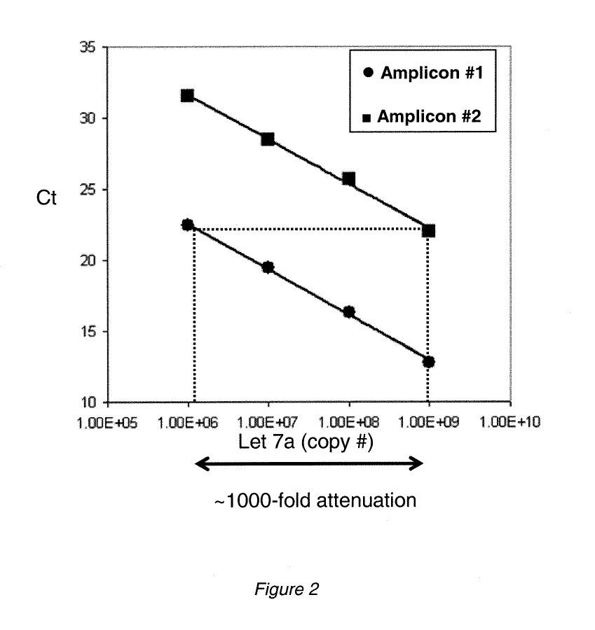

Following assembly of the reaction components on ice, 1 μl of the corresponding reverse transcription reaction was transferred to the PCR mix. PCR reactions were incubated in an ABI PRISM™ 7900HT Fast Real-Time system (Applied Biosystems) at 95° C. for 1 min, then for 40 cycles of 95° C. for 5 sec and 60° C. for 30 sec. Results shown in Table 5 and FIG. 2 demonstrate that a 5000-fold dilution of one of the RT primers compared to the other maintains a highly efficient amplification (˜100% PCR efficiency) while enabling a shift in the detection range of at least 10 Ct (threshold cycle, the number of cycles in a reaction where the amplified product reaches a statistically significant level above a baseline) or at least 1000-fold. For example, let 7a target present at 1e10 copies could be quantified in the reaction containing 0.01 nM RT-2 primer at approximately the same readout signal (here, Ct) as target present at only 1e7 copies in the 50 nM RT-1 primer reaction. Importantly, the inclusion of a dilute concentration of the RT-2 primer had no untoward effect on the efficiency of the co-amplified amplicon bearing the zip code 1 sequence. In a variation of this assay, this example may be extended to further expand the dynamic range by the use of additional RT primers, for example with unique primer binding sites and zip code sequences that are differentially diluted with respect to one another.

TABLE 5
ATTENUATED PRODUCT YIELD FROM A SINGLE RNA
TARGET USING DIFFERENT CONCENTRATIONS
OF RT PRIMERS

| | | | | | | | Ct | |
|---|---|---|---|---|---|---|---|---|
| RT-1 | RT-2 | Copies | RT-1 | RT-1 | RT-2 | RT-2 | differ- | Fold |
| (nM) | (nM) | let 7a | Ct | Slope | Ct | Slope | ence | change |
| 50 | 0.01 | NTC | 40.00 | | 35.88 | | | |
| 50 | 0.01 | 1.00E+05 | 27.69 | −3.26 | 37.69 | −3.14 | 10.00 | 1027 |
| 50 | 0.01 | 1.00E+06 | 24.55 | | 34.90 | | 10.35 | 1309 |
| 50 | 0.01 | 1.00E+07 | 21.41 | | 31.53 | | 10.12 | 1116 |
| 50 | 0.01 | 1.00E+08 | 17.95 | | 28.46 | | 10.52 | 1464 |
| 50 | 0.01 | 1.00E+09 | 15.00 | | 25.68 | | 10.68 | 1644 |
| 50 | 0.01 | 1.00E+10 | 11.40 | | 21.98 | | 10.59 | 1539 |

### Example 3

### Dynamic Range Expansion by the Addition of a Competimer Primer

An alternative to RT primer dilution, which affects product formation during processing of the RNA into cDNA, is to use unextendable PCR primers. The unextendable PCR primers affect product formation during amplification of the cDNA at the PCR step. In one method to differentially amplify the amplicons, one of the reverse primers is mixed with an excess of a primer of identical sequence that is phosphorylated at the 3′ OH group. Such “blocked” primers, also known as competimers (U.S. Pat. No. 6,057,134) are not a substrate for polymerization by DNA polymerases. As a result, the yield of PCR product is reduced accordingly. Multiplex RT reactions were prepared using two different RT primers directed against a single target as described in Example 1. The RT primer sequences are shown in Table 6 with target, zip code, and primer binding site sequences indicated as in Example 2.

TABLE 6
RT PRIMER SEQUENCES FOR EXAMPLE 3

| | | |
|---|---|---|
| RT-1 | 5′-GGTCCGACTACCCCAACAAtaccttgaaccctacagcag | SEQ ID NO: 2 |
| | agtACTATACAAC-3′ | |
| RT-2 | 5′-CAAAGGCTGCCAACATAAAATGcaagcgtaaatgcag | SEQ ID NO: 3 |
| | cgtccaACTATACAAC-3′ | |

Reverse transcription components were assembled on ice prior to the addition of the RNA template and are shown in Table 7 below.

TABLE 7
REVERSE TRANSCRIPTION REACTION COMPONENTS FOR
EXAMPLE 3

| | μl per | Final |
|---|---|---|
| Component | 10 μl rxn | Concentration |
| Nuclease-free water | 3.80 | |
| 10X RT buffer (RETROscript, Ambion) | 1.00 | 1X |
| dNTP mix (2.5 mM each) (Ambion) | 1.00 | 0.25 mM each |
| RT Primer-1 (500 nM) | 1.00 | 50 nM |
| RT Primer-2 (500 nM) | 1.00 | 50 nM |
| RNase Inhibitor (40 U/μl) (Ambion) | 0.10 | 0.4 U/μl |
| MMLV-RT (100 U/μl) (Ambion) | 0.10 | 1 U/μl |
| Synthetic let 7a RNA | 2.00 | |

Following assembly of the reaction components on ice, the RNA template (1e6-1e10 copies of a synthetic mature let 7a RNA) was added in a background of 10 ng/μl of Poly(A) RNA. The reverse transcription reaction was incubated in a 96-well GeneAmp™ PCR System 9700 (Applied Biosystems) at 16° C. for 15 min, then at 42° C. for 15 min, then at 85° C. for 10 min, then transferred to ice.

For PCR, the reaction components shown in Table 8 below were assembled on ice prior to the addition of the cDNA from the reverse transcription reaction above. To evaluate the effect of a competimer in the PCR reaction, 3′ phosphorylated reverse primer 2 (10 μM) was prepared at 3:1, 7:1, or 14:1 stochiometric ratios with the reverse primer 2 (10 μM) and 0.75 μl total of the mixed primers was added per reaction. For the no-competimer control, reverse primer 2 was added at 500 nM. Final Rev-2 Primer/competimer concentrations are shown in the Table 9 below. Sequences for the forward primer, reverse primer, competimer, and TaqMan™ probes are shown in Table 10.

TABLE 8
REAL-TIME PCR COMPONENTS FOR EXAMPLE 3

| | μl per | Final |
|---|---|---|
| Component | 15 μl rxn | Concentration |
| Nuclease-free water | 5.65 | |
| MgCl₂ (50 mM) | 1.50 | 5 mM |
| 10X Platinum PCR Buffer, Minus Mg | 1.50 | 1X |
| (Invitrogen) | | |
| dNTP mix (2.5 mM each) | 1.50 | 0.25 mM each |
| let 7a Forward 13 Primer (10 μM) | 0.75 | 500 nM |
| Rev-1 (10 μM) | 0.75 | 500 nM |
| Rev-2 + Rev-2 competimer 10 μM each, | 0.75 | 500-33.3 nM Rev |
| mixed at 1:4, 1:8, and 1:15 ratios | | 0 to 467 nM |
| | | Competimer |
| RT-1 VIC MGB TaqMan ™ Probe (2 μM) | 0.60 | 80 nM |
| RT-2 FAM TAMRA TaqMan ™ Probe | 0.60 | 80 nM |
| (2 μM) | | |
| 50X ROX Internal marker | 0.30 | 1X |
| Platinum Taq (5 U/μl) | 0.10 | 0.033 U/μl |
| cDNA from RT reaction | 1.00 | |

TABLE 9
COMPETIMER RATIOS FOR EXAMPLE 3

| | | Competimer |
|---|---|---|
| Ratio | Reverse 2 Primer Concentration | Concentration |
| No Competimer | 500 nM | 0 nM |
| 3:1 | 125 nM | 375 nM |
| 7:1 | 62.5 | 437.5 nM |
| 14:1 | 33.3 nM | 466.6 nM |

TABLE 10
PCR PRIMER SEQUENCES FOR EXAMPLE 3

| | | |
|---|---|---|
| let 7a | 5′-CGGCGCGTGAGGTAGTAGGT-3′ | SEQ ID NO: 4 |
| Forward | | |
| Primer | | |
| Rev-1 | 5′-GGTCCGACTACCCCAACAA-3′ | SEQ ID NO: 5 |
| Reverse | | |
| Primer | | |
| Rev-2 | 5′-CAAAGGCTGCCAACATAAAATG-3′ | SEQ ID NO: 6 |
| Reverse | | |
| Primer | | |
| Rev-2 | 5′-CAAAGGCTGCCAACATAAAATG-3′- | SEQ ID NO: 6 |
| Reverse | Phos | |
| Competi- | | |
| mer | | |
| RT-1 VIC | 5′-VIC-CTTGAACCCTACAGCAGAGT- | SEQ ID NO: 7 |
| MGB | MGB-NFQ-3′ | |
| Taq- | | |
| Man ™ | | |
| Probe | | |
| RT-2 FAM | 5′-FAM-CAAGCGTAAATGCAGCGT | SEQ ID NO: 8 |
| TAMRA | CCA-TAMRA-3′ | |
| Taq- | | |
| Man ™ | | |
| Probe | | |

Following assembly of the reaction components on ice, 1 μl of the corresponding reverse transcription reaction was transferred to the PCR mix. PCR reactions were incubated in a 384-well ABI PRISM™ 7900HT Fast Real-Time system (Applied Biosystems) at 95° C. for 1 min, then for 40 cycles of 95° C. for 5 sec and 60° C. for 30 sec.

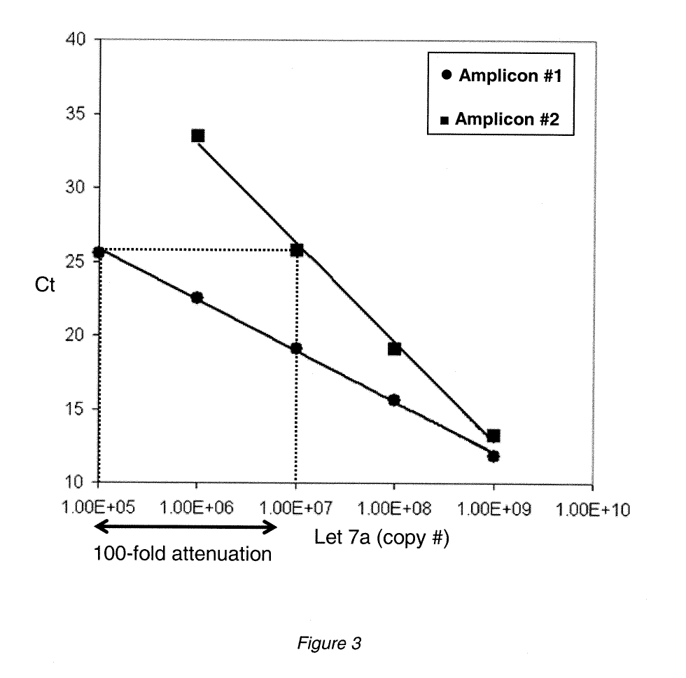

Results for the no-competimer and 3:1 ratio of competimer:Rev-2 primer are shown in Table 11. Addition of competimer and reverse primer at a 3:1 ratio increased the slope of the amplification from −3.43 to −6.47, enabling dynamic range expansion on the order of two logs or 100-fold (FIG. 3). Expansion in dynamic range was possible since the higher slope (reduced PCR efficiency) enables a broader range of input target molecules and corresponding signal responses to be compressed within into a narrower response window. Thus, the competimer-targeted amplicon was reduced in efficiency (˜43% PCR efficiency) as expected, whereas the co-amplified amplicon containing the alternative zip code—here, zip code 1—was efficiently amplified (˜96% PCR efficiency). By way of analysis, the signal difference between 1e6 and 1e8 copies input was reduced from a 6 Ct (no competimer) to <1 Ct (with 3:1 competimer:Rev-2 primer). As a result, very high signals that may saturate the detection capabilities of a particular detection platform can be reduced so that the signal is detectable within the dynamic range of the platform and appropriately quantified.

TABLE 11
ATTENUATED AMPLIFICATION OF MULTIPLE AMPLICONS
FROM A SINGLE RNA TARGET ACCOMPLISHED BY THE
ADDITION OF A COMPETIMER PRIMER

| RT | Re- | | | | | |
|---|---|---|---|---|---|---|
| Prim- | verse | Competimer | let 7a | Avg | | Ct Shift |
| er | (nM) | (nM) | copies | Ct | Slope | (RT2)-(RT-1) |
| RT-1 | 500 | 0 | NTC | 32.11 | | |
| RT-1 | 500 | 0 | 1.00E+06 | 25.58 | −3.436 | |
| RT-1 | 500 | 0 | 1.00E+07 | 22.54 | | |
| RT-1 | 500 | 0 | 1.00E+08 | 19.14 | | |
| RT-1 | 500 | 0 | 1.00E+09 | 15.60 | | |
| RT-1 | 500 | 0 | 1.00E+10 | 11.87 | | |
| RT-2 | 125 | 375 | NTC | 40 | | |
| RT-2 | 125 | 375 | 1.00E+06 | 39.71 | −6.719 | 14.13 |
| RT-2 | 125 | 375 | 1.00E+07 | 33.45 | | 10.91 |
| RT-2 | 125 | 375 | 1.00E+08 | 25.74 | | 6.60 |
| RT-2 | 125 | 375 | 1.00E+09 | 19.13 | | 3.53 |
| RT-2 | 125 | 375 | 1.00E+10 | 13.28 | | 1.41 |

### Example 4

### Dynamic Range Expansion by the Use of a Primer with Reduced Binding to Amplicons During PCR

In the following example, dynamic range expansion occurs in the PCR portion of a two-step RT-PCR reaction by the use of a reverse PCR primer with reduced capacity to bind the amplicon(s) during the PCR reaction. Although such reduced binding may be accomplished by several means, including base modifications or mismatches that lower the fraction of primer that is bound during each PCR cycle, in this example unusually short primers that have a much lower melting temperature (Tm) than that recommended for efficient PCR were evaluated. The RT primer sequences are shown in Table 12.

TABLE 12
RT PRIMER SEQUENCES FOR EXAMPLE 4

| | | |
|---|---|---|
| RT-1 | 5′-GGTCCGACTACCCCAACAAtaccttgaaccctacag | SEQ ID NO: 2 |
| | cagagtACTATACAAC-3′ | |
| RT-2 | 5′-CAAAGGCTGCCAACATAAAATGcaagcgtaaatgcag | SEQ ID NO: 3 |
| | cgtccaACTATACAAC-3′ | |

Reverse transcription components were assembled on ice prior to the addition of the RNA template and are shown in Table 13 below.

TABLE 13
REVERSE TRANSCRIPTION REACTION COMPONENTS FOR
EXAMPLE 4

| | μl per | final |
|---|---|---|
| Component | 10 μl rxn | concentration |
| Nuclease-free water | 3.80 | |
| 10X RT buffer (RETROscript, Ambion) | 1.00 | 1X |
| dNTP mix (2.5 mM each) (Ambion) | 1.00 | 0.25 mM each |
| RT Primer-1 (500 nM) | 1.00 | 50 nM |
| RT Primer-2 (500 nM) | 1.00 | 50 nM |
| RNase Inhibitor (40 U/μl) (Ambion) | 0.10 | 0.4 U/μl |
| MMLV-RT (100 U/μl) (Ambion) | 0.10 | 1 U/μl |
| Synthetic let 7a RNA | 2.00 | |

Following assembly of the reaction components on ice, the RNA template (1e5-1e11 copies of a synthetic mature let 7a RNA) was added in a background of 10 ng/μl of Poly(A) RNA. The reverse transcription reaction was incubated in a 384-well GeneAmp™ PCR System 9700 (Applied Biosystems) at 16° C. for 15 min, then at 42° C. for 15 min, then at 85° C. for 10 min, then transferred to ice.

For the PCR reaction, an amount of 1 μl of the RT reaction was transferred to a 2-plex PCR reaction (15 μl) using a common let 7a forward primer and a standard Tm reverse primer (Tm=56.8° C. by IDT OligoAnalyzer 3.0) specific to the RT-1 tail sequence and 22-base standard Rev-2 or 9-base or 8-base reduced Tm primer sequence specific to the RT-2 tail sequence. Reverse primer sequences with respective Tm values are shown in Table 14.

TABLE 14

MELTING TEMPERATURE (TM) OF REVERSE PCR PRIMERS USED IN
EXAMPLE 4

| | | |
|---|---|---|
| Primer | Sequence | Tm |

| | | | |
|---|---|---|---|
| Rev-1 Reverse Primer | 5′-GGTCCGACTACCCCAACAA-3′ | 56.8° C. | SEQ ID NO: 5 |
| Rev-2 Reverse Primer | 5′-CAAAGGCTGCCAACATAAA | 53.2° C. | SEQ ID NO: 6 |
| | ATG-3′ | | |
| Rev-2 Short Reverse | 5′-CAAAGGCTG-3′ | 25.4° C. | SEQ ID NO: 9 |
| Primer (9-mer) | | | |
| Rev-2 Short Reverse | 5′-CAAAGGCT-3′ | 17.6° C. | SEQ ID NO: 10 |
| Primer (8-mer) | | | |

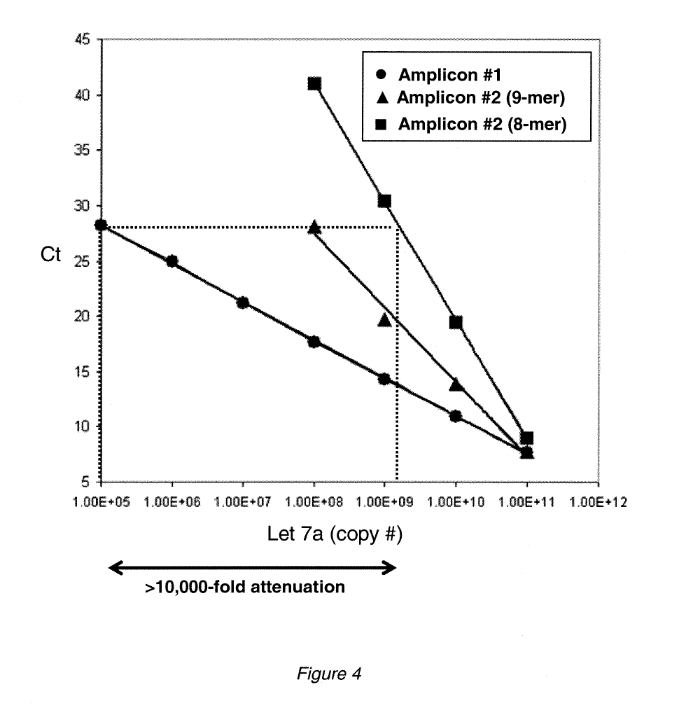

Results shown in Table 15 demonstrate that addition of 9-mer or 8-mer reverse primers in the PCR reaction significantly decreases the efficiency of the PCR reaction, resulting in a slope of the standard curve of −3.44 (conventional reverse primer length) to −6.70 (9-mer reverse primer) and −10.67 (8-mer reverse primer), respectively. Thus the addition of a 9-mer reverse primer in the PCR reaction attenuated the efficiency of the PCR amplification reaction of zip code 2 by about 1000-fold (FIG. 4) even as the other amplicon, containing zip code 1, was concurrently amplified with a PCR efficiency of ˜95%. Addition of an 8-mer reverse primer attenuated the amplification of the amplicon bearing the zip code 2 sequence more than 10,000-fold (FIG. 4), even as, again, the efficiency of the co-amplified amplicon bearing zip code 1 remained very high. In Table 15, NTC stands for non-template control, and Ct shift is calculated according to the following method: Ct, non-attenuated amplicon (RT-1), RNA input copy number x-Ct, attenuated amplicon (RT-2), RNA input copy number x-.

TABLE 15
ATTENUATED AMPLIFICATION OF MULTIPLE AMPLICONS
FROM A SINGLE RNA TARGET ACCOMPLISHED BY THE
USE OF PCR PRIMERS WITH REDUCED BINDING TO THE
TARGET AMPLICON.

| | RT | Rev-2 | Rev Tm | | | Ct Shift |
|---|---|---|---|---|---|---|
| Copies | Primer | (nt) | (° C.) | Ct | Slope | (RT-2)-(RT-1) |
| NTC | RT-1 | 22 | 56.8 | 39.52 | | |
| 1.00E+05 | RT-1 | 22 | 56.8 | 28.18 | −3.44 | |
| 1.00E+06 | RT-1 | 22 | 56.8 | 24.94 | | |
| 1.00E+07 | RT-1 | 22 | 56.8 | 21.23 | | |
| 1.00E+08 | RT-1 | 22 | 56.8 | 17.61 | | |
| 1.00E+09 | RT-1 | 22 | 56.8 | 14.26 | | |
| 1.00E+10 | RT-1 | 22 | 56.8 | 10.95 | | |
| 2.00E+11 | RT-1 | 22 | 56.8 | 7.71 | | |
| NTC | RT-2 | 9 | 25.4 | 50.00 | | |
| 1.00E+05 | RT-2 | 9 | 25.4 | 50.00 | | 21.82 |
| 1.00E+06 | RT-2 | 9 | 25.4 | 50.00 | | 21.95 |
| 1.00E+07 | RT-2 | 9 | 25.4 | 45.88 | | 10.27 |
| 1.00E+08 | RT-2 | 9 | 25.4 | 29.97 | −6.70 | 10.50 |
| 1.00E+09 | RT-2 | 9 | 25.4 | 27.78 | | 5.50 |
| 1.00E+10 | RT-2 | 9 | 25.4 | 22.47 | | 2.94 |
| 2.00E+11 | RT-2 | 9 | 25.4 | 7.12 | | 0.01 |
| NTC | RT-2 | 8 | 17.6 | 50 | | |
| 1.00E+05 | RT-2 | 8 | 17.6 | 50 | | 21.82 |
| 1.00E+06 | RT-2 | 8 | 17.6 | 50 | | 25.05 |
| 1.00E+07 | RT-2 | 8 | 17.6 | 50 | | 28.76 |
| 1.00E+08 | RT-2 | 8 | 17.6 | 40.91 | −10.67 | 23.29 |
| 1.00E+09 | RT-2 | 8 | 17.6 | 30.34 | | 16.08 |
| 1.00E+10 | RT-2 | 8 | 17.6 | 19.47 | | 8.516 |
| 2.00E+11 | RT-2 | 8 | 17.6 | 8.94 | | 1.23 |

### Example 5

### The Existing Dynamic Range of the Luminex 100 IS System is Limited

Preparation of conjugated FlexMAP™ Microspheres (Luminex) was performed as per the manufacturer's protocol producing a conjugated bead stock of approximately 50,000 microspheres/μl with approximately 1 μM capture oligonucleotide (Biosearch Technologies). Sequences of the capture oligonucleotides with the respective Luminex microsphere identification number are listed in Table 16 below and are the complementary sequence to RT-1 VIC MGB-NFQ TaqMan™ Probe and RT-2 FAM TAMRA TaqMan™ Probe as listed in Example 2 and Table 4 above, with the exception that Capture Oligo 2 was been shortened to a length of 18 nucleotides.

TABLE 16

CAPTURE OLIGONUCLEOTIDES CONJUGATED TO
FLEXMAP ™ MICROSPHERES

| | | Luminex | |
|---|---|---|---|
| Capture | | Micro- | |
| Oligo | | sphere | |
| Name | Sequence | ID | |
| Capture | 5′-CTT GAA CCC TAC | 80 | SEQ ID NO: 11 |
| Oligo 1 | AGC AGA GT-3′ | | |
| (CO-1) | | | |
| Capture | 5′-CAA GCG TAA ATG | 83 | SEQ ID NO: 12 |
| Oligo 2 | CAG CGT-3′ | | |
| (CO-2) | | | |

First, the conjugated beads were diluted 1:1000 in 0.75× TMAC Hybridization Buffer (2.25 M Tetramethylammonium chloride, 37.5 mM Tris-HCl pH 8.0, 0.075% N-Lauryl Sarkosyl, 3 mM EDTA pH 8.0). Then 45 μl of each bead type was functionally tested in a hybridization reaction with a range of respective biotinylated complement oligonucleotide from 0, 1, 2, 5, 10, 25, 50, 75, 100, 150, 200, and 1000 fmol (5 μl each). The 50-μl hybridization reaction was incubated for 95° C. for 5 min then 52° C. for 25 min in a 96-well GeneAmp™ PCR System 9700 (Applied Biosystems). Immediately following hybridization, 500 ng of Phycolink™ Streptavidin R-Phycoerythrin (PROzyme) prepared in 0.75× TMAC Hybridization Buffer was added to the reaction then the fluorescence signal was detected by flow cytometry on the Luminex 100 IS System with the following Advanced Batch Settings: Sample size=50 μl, DD Gate=7500 to 19000, Timeout=25 sec, 100 Regions, and Bead Events=100 Total Beads. Hybridization was performed in both simplex and duplex, where a simplex reaction contained ˜225,000 conjugated beads of a single microsphere ID and the duplex condition contained a mixture of both CO-1 and CO-2 (˜225,000 of each microsphere). Results in Table 17 show similar median fluorescent intensity (MFI) values for both CO-1 and CO-2 simplex and duplex conditions. Taken together, the data reveal that the highest input of CO-1 that yields a dose-dependent signal is ˜25 fmol. The lowest input that would produce a signal appreciably above background (e.g., two times signal to noise ratio) can be extrapolated from the linear portion of the curve; this input would correspond to ˜0.25 fmol. Thus the instrument dynamic range is approximately 100-fold, or ˜two logs. Comparable results are obtained after inspection of the CO-2 data.

TABLE 17
MFI VALUES FOR SIMPLEX AND DUPLEX HYBRIDIZATION CONDITIONS
Average MFI

| Biotinylated | Microsphere | Amount of Biotin Complement Oligo (fmol) | | | | | | | | | | | |
|---|---|---|---|---|---|---|---|---|---|---|---|---|---|
| Oligo | Mix | 1000 | 200 | 150 | 100 | 75 | 50 | 25 | 10 | 5 | 2 | 1 | 0 |
| CO-1 Detection | | | | | | | | | | | | | |
| CO-1 | CO-1 | 19827 | 20635 | 20818 | 20811 | 20741 | 20305 | 18742 | 12779 | 8411 | 3953 | 2358 | 245 |
| Complement | | | | | | | | | | | | | |
| CO-1 | CO-1 and | 18764 | 18761 | 20230 | 20813 | 20126 | 19908 | 18460 | 12309 | 7360 | 3850 | 2265 | 269 |
| Complement | CO-2 | | | | | | | | | | | | |
| CO-1 | CO-1 and | 17577 | 20394 | 20088 | 20830 | 20760 | 20859 | 18568 | 13047 | 7705 | 3908 | 2147 | 230 |
| and CO-2 | CO-2 | | | | | | | | | | | | |
| Complement | | | | | | | | | | | | | |
| CO-2 Detection | | | | | | | | | | | | | |
| CO-2 | CO-2 | 18342 | 19660 | 19991 | 20220 | 20204 | 18863 | 15053 | 11035 | 6543 | 4226 | 2504 | 753 |
| Complement | | | | | | | | | | | | | |
| CO-2 | CO-1 and | 18859 | 20145 | 20482 | 20814 | 19939 | 19842 | 14913 | 10940 | 6486 | 3987 | 2388 | 638 |
| Complement | CO-2 | | | | | | | | | | | | |
| CO-1 | CO-1 and | 16358 | 20015 | 20603 | 20579 | 19395 | 18765 | 14653 | 10529 | 6176 | 4415 | 2282 | 720 |
| and Co-2 | CO-2 | | | | | | | | | | | | |
| Complement | | | | | | | | | | | | | |

### Example 6

### Methods for Competitive Hybridization on the Luminex

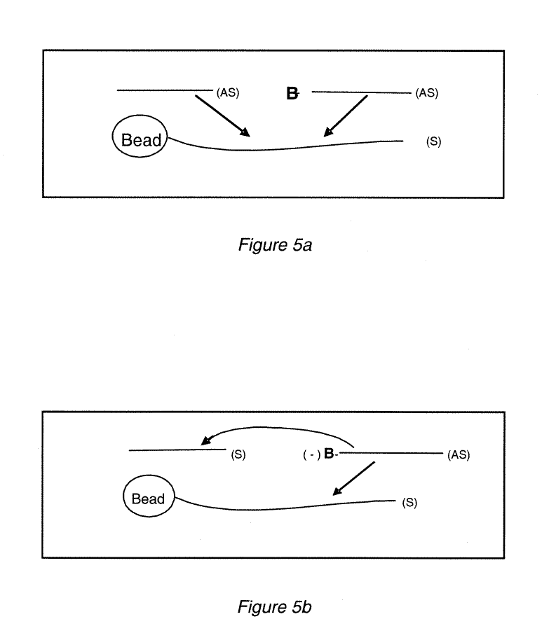

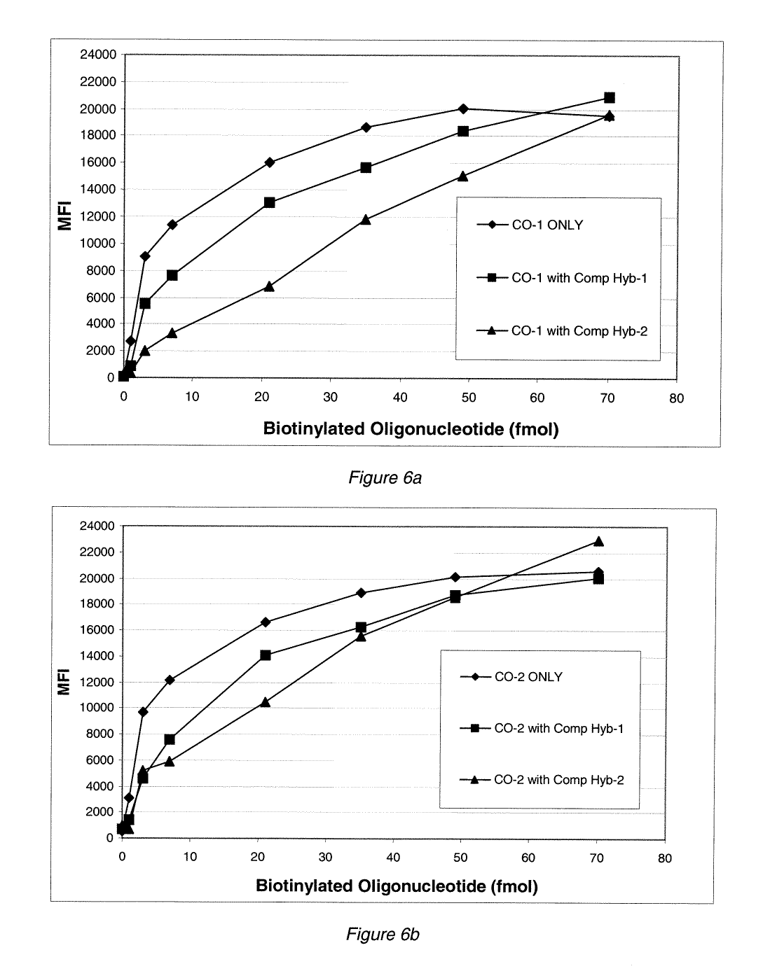

As demonstrated in Example 5, the Luminex platform is only capable of ˜two logs of dynamic range. This range of dose-dependent signal response is inadequate for many clinical assays. As an alternative (or in addition) to attenuated amplification (Examples 2-4), attenuated hybridization may be used to titrate saturating signals into the responsive range of the instrument. To evaluate attenuated hybridization, competing oligonucleotides were added in one of two configurations during the hybridization step: 1) a non-biotinylated oligo with a complementary sequence to the capture oligonucleotide (Comp Hyb-1 as shown in FIG. 5a), or 2) a non-biotinylated oligo with a sequence identical to the capture oligo (Comp Hyb-2 as shown in FIG. 5b). In the first scenario, the non-biotinylated oligo competes with the biotinylated oligo for the capture oligo. In the second scenario, signal attenuation occurs as the non-biotinylated oligo and the capture oligo compete for the biotinylated oligo. To evaluate these exemplary approaches to attenuated hybridization, biotinylated and non-biotinylated oligos were combined in the following ratios in 5 μl: 0:0, 1:69, 3.5:66.5, 7:63 21:49, 35:35 49:21 70:0 fmol. A total of 45-μl of conjugated microspheres previously diluted 1:1000 in 0.75× TMAC Hybridization Buffer, was then hybridized and detected on the Luminex as described in Example 5. The results in FIGS. 6a and 6b reveal that the competitive hybridization increased the dynamic range with both oligonucleotides. Comp Hyb-2 show a more linear response.

### Example 7

### Multiplexed Dose Dependence of Comp Hyb-2 on CO-2 Signal in a Background of Non-Attenuated CO-1

The following example demonstrates the utility of competitive hybridization. This example illustrates that the addition of a non-biotinylated oligo as a competitor of the target molecule (see Comp Hyb-2 configuration described in Example 6) attenuates the MFI signal of CO-2 in a background of CO-1. Detection of CO-1 is not affected. In this case, the biotin and competing non-biotinylated oligo were prepared as follows in nuclease-free water: 0, 0.1, 0.5, 0.75, 1.0, 1.5, and 2.0 pmol biotinylated oligo with either 0, 50, 100, 200, 300, or 400 pmol non-biotinylated oligo. As a result, ratios of biotin:non-biotin oligo were created ranging from 1:25 to 1:4000 to observe the effects of attenuation. A duplex conjugated microsphere mixture was prepared, hybridized, and detected as described in Example 5. The results in Table 18 below indicate the most linear response occurs with 100 pmol non-biotinylated oligo (R²=0.9802), and the least linear response with 0 pmol non-biotinylated oligo (R²=0.3241). Table 19 shows the MFI values collected for CO-1, which contained 1 pmol biotinylated oligo only.

TABLE 18
MFI VALUES FOR CO-2 WITH COMPETITIVE HYBRIDIZATION

| | Non-Biotin Competimer pmol | | | | | |
|---|---|---|---|---|---|---|
| Biotin pmol | 0 | 400 | 300 | 200 | 100 | 50 |
| 2.00 | 15610 | 103 | 364 | 758 | 1208 | 2333 |
| 1.50 | 15632 | 218 | 426 | 480 | 944 | 2176 |
| 1.00 | 17124 | 92 | 205 | 416 | 613 | 1025 |
| 0.75 | 17046 | 112 | 63 | 237 | 447 | 776 |
| 0.50 | 17333 | −33 | 140 | 151 | 175 | 362 |
| 0.10 | 890 | −97 | 58 | 69 | 78 | 123 |
| R2 Values | 0.3241 | 0.6742 | 0.7610 | 0.9519 | 0.9802 | 0.9351 |

TABLE 19
MFI VALUES FOR CO-1 WITH NON-COMPETITIVE
HYBRIDIZATION

| Biotin | 1 pmol Biotinyated Oligo with CO-1 | | | | | |
|---|---|---|---|---|---|---|
| pmol | 0 | 400 | 300 | 200 | 100 | 50 |
| 2.00 | 15266 | 15249 | 17192 | 18256 | 18695 | 20074 |
| 1.50 | 17415 | 16825 | 18716 | 19109 | 18926 | 19416 |
| 1.00 | 18531 | 19889 | 19326 | 20630 | 20572 | 20686 |
| 0.75 | 18898 | 20747 | 20137 | 21230 | 21207 | 20875 |
| 0.50 | 19297 | 20341 | 19988 | 20773 | 21129 | 20952 |
| 0.10 | 20165 | 20251 | 21239 | 21188 | 21314 | 21259 |

### Example 8

### Attenuation of CO-2 Signal into a Detectable Range in a Background of CO-1

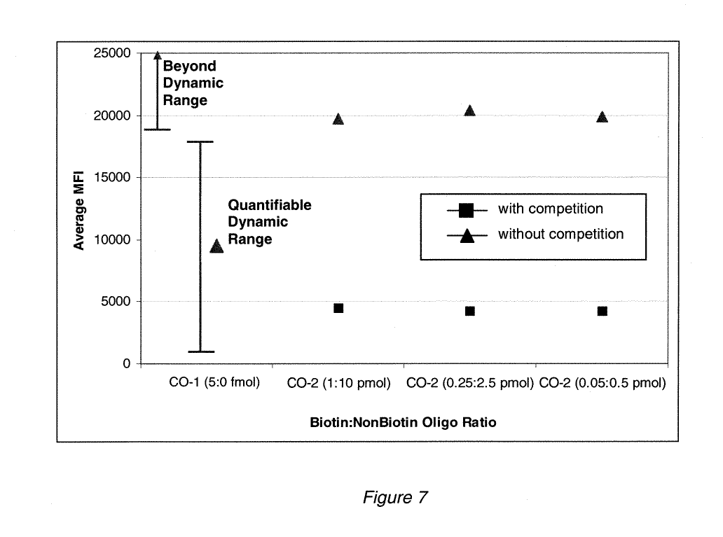

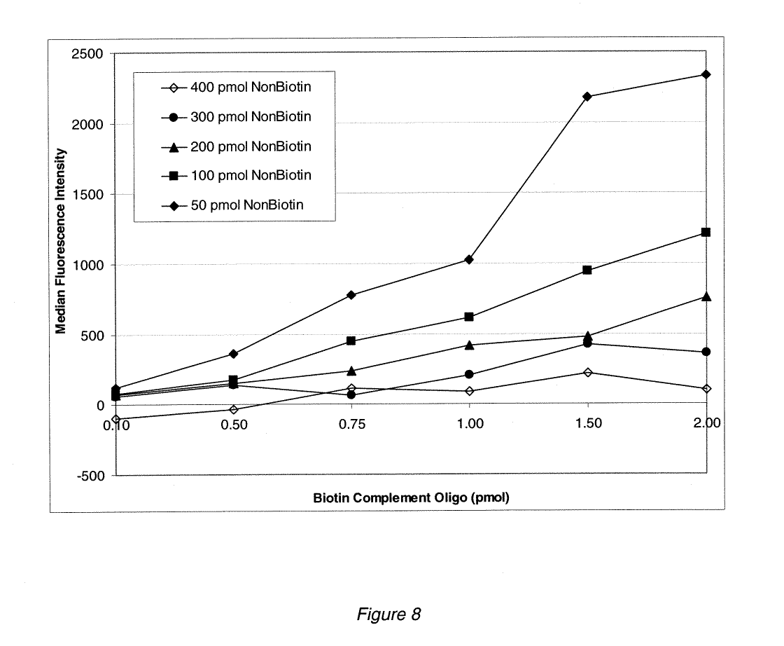

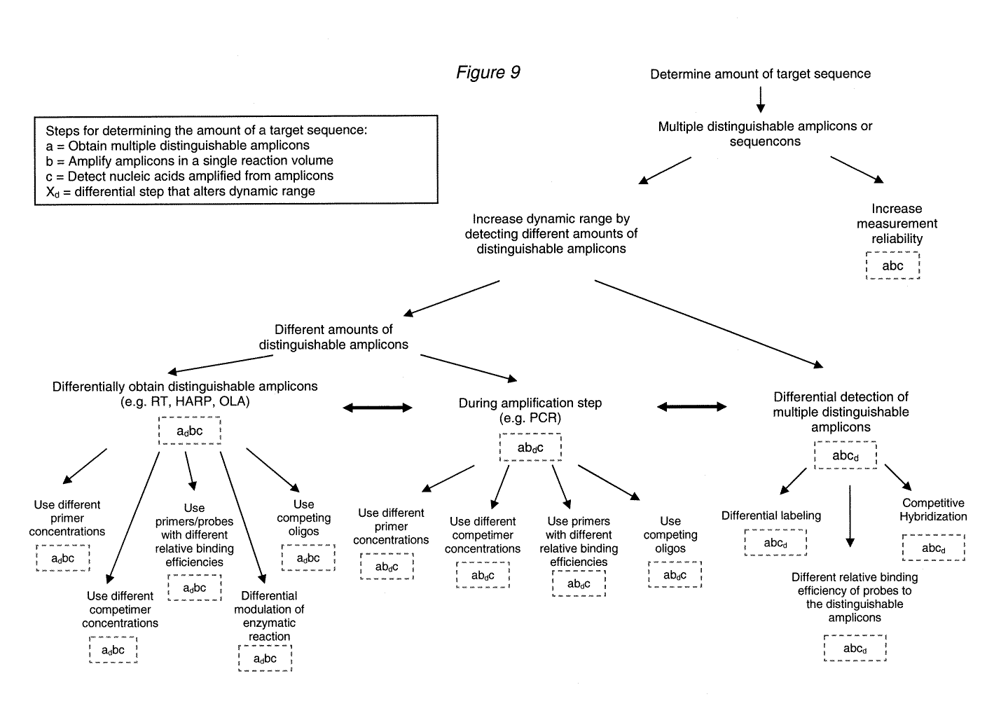

The following example demonstrates the attenuation of CO-2 MFI values by competitive hybridization into a detectable and quantifiable range of CO-1. Using the preferred method of competitive hybridization identified in Example 7, Comp Hyb-2 with CO-2, reactions were prepared at a ratio of 1:10 of biotinylated and non-biotinylated oligo as follows: 1:10 pmol, 0.25:2.5 pmol, and 0.05:0.5 pmol, each in a background of 5 fmol CO-1. The multiplexed assay was hybridized and data collected as previously described in Example 5. In FIG. 7 below, the MFI values for the attenuated reactions for CO-2 were readily detected within the quantifiable range of CO-1 (solid squares). In contrast, the MFI values for the unattenuated CO-2 reactions were well beyond the quantifiable dynamic range of CO-1 (solid triangles). Thus a common target sequence can be detected through unique “zip code” tag sequences (the sequences described in Examples 5-8). The individual amplicons represent different levels of attenuation at the hybridization step to accommodate a much broader range of quantifiable signals. For example, as shown in FIG. 7, as little as 5 fmol (no attenuation) and as much as 1 pmol (10× attenuation with competing, non-biotinylated oligo) can produce roughly comparable signals. Thus, at least a 1000-fold range of inputs can be quantified by the method of the invention, a breadth that far exceeds the nominal ˜100-fold instrument dynamic range. Moreover, multiple competing zip code tag sequences may be used with differential levels of their respective non-labeled oligo to span an even broader range. In this case, the only limitation is that the windows of attenuation provided by each zip coded amplicon overlap with the fundamental instrument dynamic range response, i.e., within two logs of one another. Last, attenuated hybridization may be combined with attenuated amplification to accommodate the broadest range of target inputs.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth

The specification is most thoroughly understood in light of the teachings of the references cited within the specification which are hereby incorporated by reference. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. The citation of any references herein is not an admission that such references are prior art to the present invention. The representations of molecular mechanisms and pathways are provided for ease of the understanding of the invention only and should not be considered binding. The specification and examples are to be considered as exemplary only, with a true scope and spirit of the invention being indicated by the following claims.

What is claimed is:

1. A method of determining the amount of a target nucleic acid sequence in a sample, the method comprising:
(a) obtaining multiple distinguishable amplicons of the target nucleic acid sequence, each comprising a distinguishing tag and a target portion, wherein each target portion is complementary to an identical target nucleic acid subsequence or its complement;
(b) amplifying the amplicons in a single reaction volume; and
(c) detecting nucleic acids amplified from at least two distinguishable amplicons.

2. A method of determining the amount of a target nucleic acid sequence in a sample, the method comprising:
(a) obtaining multiple distinguishable amplicons of the target nucleic acid sequence, each comprising a distinguishing tag and a target portion, wherein the target portion is identical in sequence and length in each distinguishable amplicon;
(b) amplifying the amplicons in a single reaction volume; and
(c) detecting nucleic acids amplified from at least two distinguishable amplicons.

3. The method of claim 1, further comprising comparing the amount of the nucleic acids detected in part (c), thereby increasing target measurement reliability.

4. The method of claim 1, wherein different amounts of at least two distinguishable amplicons are detected, thereby increasing the dynamic range of the method of determining the amount of the target.

5. The method of claim 1, wherein the target portion of the multiple distinguishable amplicons is completely complementary to the target nucleic acid subsequence.

6. The method of claim 1, wherein the multiple distinguishable amplicons are obtained in part (a) in different amounts, thereby increasing the dynamic range of the method of determining the amount of the target.

7. The method of claim 6, wherein the multiple distinguishable amplicons are obtained in different amounts using different concentrations of primers to the target nucleic acid sequence.

8. The method of claim 6, wherein the multiple distinguishable amplicons are obtained in different amounts using different concentrations of competimers to the target nucleic acid sequence.

9. The method of claim 6, wherein the multiple distinguishable amplicons are obtained in different amounts using two or more primers that have different relative binding efficiencies to the target nucleic acid sequence.

10. The method of claim 9, wherein the primers comprise a modified nucleotide.

11. The method of claim 9, wherein the primers comprise a mismatch to the target nucleic acid sequence.

12. The method of claim 9, wherein the primers have different lengths.

13. The method of claim 9, wherein the primers bind to the target nucleic acid sequence with different melting temperatures.

14. The method of claim 6, wherein the multiple distinguishable amplicons are obtained in different amounts using at least one oligonucleotide that competes with the target nucleic acid sequence for binding to a distinguishable primer.

15. The method of claim 6, wherein the multiple distinguishable amplicons are obtained by a method comprising:
(a) contacting the sample containing the target nucleic acid sequence with multiple distinguishable primers under hybridization conditions, wherein the multiple distinguishable primers comprise:
(i) a target binding sequence; and
(ii) a distinguishing tag; and
(b) producing multiple distinguishable amplicons in a primer-dependent enzymatic reaction.

16. The method of claim 6, wherein the multiple distinguishable amplicons are obtained by a method comprising:
(a) contacting the sample containing the target nucleic acid sequence with multiple distinguishable probes under hybridization conditions, wherein the multiple distinguishable probes comprise:
(i) a target binding sequence; and
(ii) a distinguishing tag; and
(b) adding a modifying agent to differentiate non-hybridized and hybridized probes.

17. The method of claim 1, wherein the multiple distinguishable amplicons are amplified at different amplification efficiencies, thereby increasing the dynamic range of the method of determining the amount of the target.

18. The method of claim 17, wherein the multiple distinguishable amplicons are amplified using different concentrations of primers to at least two distinguishable amplicons.

19. The method of claim 17, wherein the multiple distinguishable amplicons are amplified using at least one competimer to a distinguishable amplicon.

20. The method of claim 17, wherein the multiple distinguishable amplicons are amplified using two or more primers that have different relative binding efficiencies to at least two distinguishable amplicons.

21. The method of claim 20, wherein the primers comprise a modified nucleotide.

22. The method of claim 20, wherein the primers comprise a mismatch to the target nucleic acid sequence.

23. The method of claim 20, wherein the primers have different lengths.

24. The method of claim 20, wherein the primers bind to the distinguishable amplicons with different melting temperatures.

25. The method of claim 17, wherein the multiple distinguishable amplicons are amplified using at least one oligonucleotide that competes with a distinguishable amplicon for binding to a primer.

26. The method of claim 17, wherein at least two distinguishable amplicons are amplified to different amounts of single stranded nucleic acid products.

27. The method of claim 1, wherein the nucleic acids amplified from the multiple distinguishable amplicons are differentially detected in part (c), thereby increasing the dynamic range of the method of determining the amount of the target.

28. The method of claim 27, wherein the amplified nucleic acids are detected by competitive hybridization with probes for the distinguishable amplicons.

29. The method of claim 28, further comprising using a nucleic acid sequence that competes for binding to a distinguishable amplicon.

30. The method of claim 28, further comprising using a nucleic acid sequence that competes for binding to a probe to a distinguishable amplicon.

31. The method of claim 27, wherein the probes or primers are differentially labeled.

32. The method of claim 27, wherein the amplified nucleic acids are detected by probes that bind to at least two distinguishable amplicons with different relative binding efficiencies.

33. The method of claim 32, wherein the probes bind to the distinguishable amplicons with different melting temperatures.

34. The method of claim 27, wherein the amplified nucleic acids are detected with probes having different single stranded versus double stranded character.

35. A method of determining the amount of a target nucleic acid sequence in a sample, the method comprising:
(a) obtaining multiple distinguishable sequencons of the target nucleic acid sequence in a single reaction volume, each comprising a distinguishing tag and a target portion, wherein each target portion is complementary to an identical target nucleic acid subsequence or its complement; and
(b) detecting different amounts of least two of the sequencons, thereby increasing the dynamic range of the method of determining the amount of the target nucleic acid.

36. A method of determining the amount of a target nucleic acid sequence in a sample, the method comprising:
(a) obtaining multiple distinguishable amplicons of the target nucleic acid sequence, each comprising a zip code and a target portion, wherein the target portions are complementary (vs. hybridize) to an identical or overlapping target nucleic acid subsequence;
(b) amplifying the amplicons in a single reaction volume; and
(c) detecting nucleic acids amplified from each of the amplicons.

37. A kit for detecting a target nucleic acid sequence in a sample comprising multiple distinguishing tags, wherein at least two of the distinguishing tags comprise:
(a) a target binding sequence that is complementary to an identical or overlapping portion of the target nucleic acid sequence; and
(b) a zip code that uniquely identifies the distinguishing tag.

38. The kit of claim 37, wherein at least two tags further comprise a primer binding site.

39. The kit of claim 37, wherein at least two tags comprise a distinguishable primer binding site.

40. The kit of claim 37 further comprising multiple amplification primer sets, wherein at least one of the primers in each of the primer sets comprises a sequence that is complementary to a portion of at least one distinguishing tag.

41. The kit of claim 37 further comprising at least two probes complementary to a portion of at least two distinguishing tags.

42. The kit of claim 37, further comprising standards for determining the amount of the target sequence.

43. The kit of claim 37, further comprising enzymes for obtaining multiple distinguishable amplicons, each comprising a portion identical or complementary to a target nucleic acid subsequence.

44. A method for determining the amount of a target nucleic acid sequence in a sample, the method comprising:
(a) producing multiple distinguishable amplicons in a primer-dependent enzymatic reaction, wherein at least two amplicons comprise a distinguishing tag and a target portion that is complementary to an identical or overlapping target nucleic acid subsequence or its complement;
(b) differentially amplifying the amplicons in a single reaction volume; and
(c) detecting nucleic acids amplified from at least two distinguishable amplicons.

45. The method of claim 44, wherein the multiple distinguishable amplicons are amplified using two or more primers that have different relative binding efficiencies to at least two distinguishable amplicons.


SEQUENCE LISTING (12 sequences)

SEQ ID NO: 1 - RNA, 22 nt, Homo sapiens
ugagguaguagguuguauaguu

SEQ ID NO: 2 - DNA, 52 bp, Artificial
  Synthetic primer
ggtccgactaccccaacaataccttgaaccctacagcagagtactatacaac

SEQ ID NO: 3 - DNA, 53 bp, Artificial
  Synthetic primer
caaaggctgccaacataaaatgcaagcgtaaatgcagcgtccaactatacaac

SEQ ID NO: 4 - DNA, 20 bp, Homo sapiens
cggcgcgtgaggtagtaggt

SEQ ID NO: 5 - DNA, 19 bp, Artificial
  Synthetic primer
ggtccgactaccccaacaa

SEQ ID NO: 6 - DNA, 22 bp, Artificial
  Synthetic primer
caaaggctgccaacataaaatg

SEQ ID NO: 7 - DNA, 20 bp, Artificial
  Synthetic primer
cttgaaccctacagcagagt

SEQ ID NO: 8 - DNA, 21 bp, Artificial
  Synthetic primer
caagcgtaaatgcagcgtcca

SEQ ID NO: 9 - DNA, 9 bp, Artificial
  Synthetic primer
caaaggctg

SEQ ID NO: 10 - DNA, 8 bp, Artificial
  Synthethic primer
caaaggct

SEQ ID NO: 11 - DNA, 20 bp, Artificial
  Synthetic probe
cttgaaccctacagcagagt

SEQ ID NO: 12 - DNA, 18 bp, Artificial
  Synthetic probe
caagcgtaaatgcagcgt